SEQUENCE LISTING (56 sequences)

SEQ ID NO: 1 - protein, 316 aa, Zea mays
MERWGDKDRGAAVPAPGRLRRYADQPSFSSSLLDAIYKSMDEPGDGATSAAAAGATKMQSHQDLHYSYYYKTSLAGSYRGSRAAAAAHAATTTSSSSECSSYGGFSSSEAESSQHRRLRPIRTSVGAAASPAPAPEKKKKAGANIRAKLRDLRKPASPGARLAGFLNTIFSGRRAPATPPSRGAESSACSTASSYSRSCLSKTPSTRGQPKRTVRFLDSDDGEAAAAAPGGERRRVQVGVAELERMLLHRMEMDSDEDDEDEEGSDASSDLFDLENFAAGAPDAAAAYRDELPVYETTRVVLGHRAIGHGRSARVV

SEQ ID NO: 2 - DNA, 951 bp, Zea mays
atggagaggtggggggacaaggacaggggggcggcggttccggcgccggggaggttgaggcggtacgccgaccagccgtccttctcgtcctcgctcctcgacgccatatacaagtccatggacgagcccggcgacggggcaacatccgccgccgccgcgggagcgaccaagatgcagagccaccaggacctgcactacagctactactacaagacgtcgctggcggggagctaccgcggcagcagggctgcggcggcggcgcacgccgccaccaccacgtcgagctcctccgaatgctcgagctacggtgggttctcgtcgtcggaggcggagtcgtcgcagcaccggcggctgcggcccatacgcacgagcgtgggcgcggccgcgtcgcccgcgcccgcgccggagaagaagaagaaggccggcgccaacatccgcgccaagctgagggacctccgcaagccggcgtcccccggcgcgcggctggcggggttcctgaacaccatcttcagcggcaggcgcgcgccggcgacgccgccgtcgcggggcgcggagtcctccgcgtgctccacggcgtcctcgtactcgcgctcctgcctcagcaagacgccgtccacgcggggccagccgaagcggaccgtgcggttcctggacagcgacgacggcgaggcggcggcggcggcgcccggcggcgagcggcggagggtgcaggtcggggtggcggagctggagcggatgctgctccaccggatggagatggacagcgacgaggacgacgaggacgaggagggcagcgacgccagctccgacctgttcgacctcgagaacttcgcggccggtgcccccgacgccgcggcggcgtacagggacgagctgccggtgtacgagacgacgagggtggtgctgggacaccgcgccattggccacgggaggagtgccagggtggtgtga

SEQ ID NO: 3 - protein, 324 aa, Zea mays
MERWGDKDRGAAVPAPGRLRRYADQPSFSSSLLDAIYKSMDEPGDGATSAAAGATKMQSQDLHYSYYYKTSLAGSYRGSRAVSAAHAATTTSSSSECSSYGGFSSSEAESSQHRRLRPIRTSVGAAASPAPAPAPAPEKKKKAGANIRAKLRDLRKPASPGARLAGFLNTIFSGRRAPATPPSRGAESSACSTASSYSRSCLSKTPSTRGQPKRTVRFLDSDDGEAAAAAPGGERRRVQVGVAELERMLLHRMEMDSDEDDEDEEGSDASSDLFDLENFAAGAPDAAAAAAYRDELPVYETTRVVLGHRAIGHGYGHGRSARVV

SEQ ID NO: 4 - DNA, 975 bp, Zea mays
atggagaggtggggggacaaggacaggggggcggcggttccggcgccggggaggttgaggcggtacgccgaccagccgtccttctcgtcctcgctcctcgacgccatatacaagtccatggacgagcccggcgacggggcaacatccgccgccgcgggagcgaccaagatgcagagccaggacctgcactacagctactactacaagacgtcgctggcggggagctaccgcggcagcagggcggtgtcggcggcacacgccgccaccaccacgtcgagctcctccgaatgctcgagctacggtgggttctcgtcgtcggaggcggagtcgtcgcagcaccggcggctgcggcccatacgcacgagcgtgggcgcggccgcgtcgcccgcgcccgcgccggcgcccgcgccggagaagaagaagaaggccggcgccaacatccgcgccaagctgagggacctgcgcaagccggcgtcccccggcgcgcggctggcggggttcctgaacaccatcttcagcggcaggcgcgcgccggcgacgccgccgtcgcggggcgccgagtcctccgcgtgctccacggcgtcctcgtactcgcgctcctgcctcagcaagacgccgtccacgcggggccagccgaagcggaccgtgcggttcctggacagcgacgacggcgaggcggcggcggcggcgcccggcggcgagcggcggagggtgcaggtcggggtggcggagctggagcggatgctgctccaccggatggagatggacagcgacgaggacgacgaggacgaggaaggcagcgacgccagctccgacctgttcgacctcgagaacttcgcggccggtgcccccgacgccgccgccgcggcggcgtacagggacgagctgccggtgtacgagacgacgagggtggtgctgggccaccgcgccattggccacgggtacgggcacgggaggagtgccagggtggtgtga

SEQ ID NO: 5 - protein, 328 aa, Zea mays
MERWGDKDRGAAVPAPGRLRRYADQPSFSSSLLDAIYKSMDEPGDGATSAAAAGATKMQSHQDLHYSYYYKTSLAGSYRGSRAAAAAAHAATTTSSSSECSSYGGFSSSEAESSQHRRLRPIRTSVGAAASPAPAPAPAPEKKKKAGANIRAKLRDLRKPASPGARLAGFLNTIFSGRRAPATPPSRGAESSACSTASSYSRSCLSKTPSTRGQPKRTVRFLDSDDGEAAAAAAPGGERRRVQVGVAELERMLLHRMEMDSDEDDEDEEGSDASSDLFDLENFAAGAPDAAAAYRDELPVYETTRVVLGHRAIGHGYGHGHGRSARVV

SEQ ID NO: 6 - DNA, 987 bp, Zea mays
atggagaggtggggggacaaggacaggggggcggcggttccggcgccggggaggttgaggcggtacgccgaccagccgtccttctcgtcctcgctcctcgacgccatatacaagtccatggacgagcccggcgacggagcaacatccgccgccgccgcgggagcgaccaagatgcagagccaccaggacctgcactacagctactactacaagacgtcgctggcggggagctaccgcggcagcagggctgcggcggcggcggcacacgccgccaccactacgtcgagctcctccgaatgctcgagctacggtgggttctcgtcgtcggaggcggagtcgtcgcagcaccggcggctgcggcccatacgcacgagcgtgggcgcggccgcgtcgcccgcgcccgcgccggcgcccgcgccggagaagaagaagaaggccggcgccaacatccgcgccaagctgagggacctgcgcaagccggcgtcccccggcgcgcggctggcggggttcctgaacaccatcttcagcggcaggcgcgcgccggcgacgccgccgtcgcggggcgcggagtcctccgcgtgctccacggcgtcgtcgtactcgcgctcctgcctcagcaagacgccgtccacgcggggccagccgaagcggaccgtgcggttcctggacagcgacgacggcgaggcggcggcggcggcggcgcccggcggcgagcggcggagggtgcaggtcggggtggcggagctggagcggatgctgctccaccggatggagatggacagcgacgaggacgacgaggacgaggagggcagcgacgccagctccgacctgttcgacctcgagaacttcgcggccggtgcccccgacgccgcggcggcgtacagggacgaactgccggtgtacgagacgacgagggtggtgctgggccaccgcgccattggccacgggtacgggcacgggcacgggaggagtgccagggtggtgtga

SEQ ID NO: 7 - protein, 327 aa, Zea mays
MERWGDKDRGAAVPAPGRLRRYADQPSFSSSLLDAIYKSMDEPGDGATSAAAAGATKMQSHQDLHYSYYYKTSLAGSYRGSRAAAAAHAATTTSSSSECSSYGGFSSSEAESSQHRRLRPIRTSVGAAASPAPAPAPAPEKKKKAGANIRAKLRDLRKPASPGARLAGFLNTIFSGRRAPATPPSRGAESSACSTASSYSRSCLSKTPSTRGQPKRTVRFLDSDDGEAAAAAAPGGERRRVQVGVAELERMLLHRMEMDSDEDDEDEEGSDASSDLFDLENFAAGGAPDAAAAAYRDELPVYETTRVVLGHRAIGHGYGHGRSARVV

SEQ ID NO: 8 - DNA, 984 bp, Zea mays
atggagaggtggggggacaaggacaggggggcggcggttccggcgccggggaggttgaggcggtacgccgaccagccgtccttctcgtcctcgctcctcgacgccatatacaagtccatggacgagcccggcgacggggcaacatccgccgccgccgcgggagcgaccaagatgcagagccaccaggacctgcactacagctactactacaagacgtcgctggcggggagctaccgcggcagcagggctgcggcggcggcgcacgccgccaccaccacgtcgagctcctccgaatgctcgagctacggtgggttctcgtcgtcggaggcggagtcgtcgcagcaccggcggctgcggcccatacgcacgagcgtgggcgcggccgcgtcgcccgcgcccgcgccggcgcccgcgccggagaagaagaagaaggccggcgccaacatccgcgccaagctgagggacctgcgcaagccggcgtcccccggcgcgcggctggcggggttcctgaacaccatcttcagcggcaggcgcgcgccggcgacgccgccgtcgcggggcgccgagtcctccgcgtgctccacggcgtcctcgtactcgcgctcctgcctcagcaagacgccgtccacgcggggccagccgaagcggaccgtgcggttcctggacagcgacgacggcgaggcggcggcggcggcggcgcccggcggcgagcggcggagggtgcaggtcggggtggcggagctggagcggatgctgctccaccggatggagatggacagcgacgaggacgacgaggacgaggaaggcagcgacgccagctccgacctgttcgacctcgagaacttcgcggccggcggtgcccccgacgccgccgcggcggcgtacagggacgagctgccggtgtacgagacgacgagggtggtgctgggccaccgcgccattggccacgggtacgggcacgggaggagtgccagggtggtgtga

SEQ ID NO: 9 - protein, 325 aa, Zea mays
MERWGDKDRGAAVPAPGRLRRYADQPSFSSSLLDAIYKSMDEPGDGATSAAAGATKMQSQDLHYSYYYKTSLAGSYRGSRAVSAAHAATTTSSSSECSSYGGFSSSEAESSQHRRLRPIRTSVGAAASPAPAPAPAPEKKKKAGANIRAKLRDLRKPASPGARLAGFLNTIFSGRRAPATPPSRGAESSACSTASSYSRSCLSKTPSTRGQPKRTVRFLDSDDGEAAAAAAPGGERRRVQVGVAELERMLLHRMEMDSDEDDEDEEGSDASSDLFDLENFAAGAPDAAAAAAYRDELPVYETTRVVLGHRAIGHGYGHGRSARVV

SEQ ID NO: 10 - DNA, 978 bp, Zea mays
atggagagatggggggacaaggacaggggggcggcggttccggcgccggggaggttgaggcggtacgccgaccagccgtccttctcgtcctcgctcctcgacgccatatacaagtccatggacgagcccggcgacggggcaacatccgccgccgcgggagcgacgaagatgcagagccaggacctgcactacagctactactacaagacgtcgctggcggggagctaccgcggcagcagggcggtgtcggcggcacacgccgccaccaccacgtcgagctcctccgaatgctcgagctacggtgggttctcgtcgtcggaggcggagtcgtcgcagcaccggcggctgcggcccatacgcacgagcgtgggcgcggccgcgtcgcccgcgcccgcgccggcgcccgcgccggagaagaagaagaaggccggcgccaacatccgcgccaagctgagggacctgcgcaagccggcgtcccccggcgcgcggctggcggggttcctgaacaccatcttcagcggcaggcgcgcgccggcgacgccgccgtcgcggggcgccgagtcctcggcgtgctccacggcgtcctcgtactcgcgctcctgcctcagcaagacgccgtccacgcggggccagccgaagcggaccgtgcggttcctggacagcgacgacggcgaggcggcggcggcggcggcgcccggcggcgagcggaggagggtgcaggtcggggtggcggagctggagcggatgctgctccaccggatggagatggacagcgacgaggacgacgaggacgaggaaggcagcgacgccagctccgacctgttcgacctcgagaacttcgcggccggtgcccccgacgccgccgccgcggcggcgtacagggacgagctgccggtgtacgagacgacgagggtggtgctgggccaccgcgccattggccacgggtacgggcacgggaggagtgccagggtggtgtga

SEQ ID NO: 11 - protein, 316 aa, Zea mays
MERWGDKDRGAAVPAPGRLRRYADQPSFSSSLLDAIYKSMDEPGDGATSAAAAGATKMQSHQDLHYSYYYKTSLAGSYRGSRAAAAAHAATTTSSSSECSSYGGFSSSEAESSQHRRLSPIRTSVGAAASPAPAPEKKKKAGANIRAKLRDLRKPASPGARLAGFLNTIFSGRRAPATPPSRGAESSACSTASSYSRSCLSKTPSTRGQPKRTVRFLDSDDGEAAAAAPGGERRRVQVGVAELERMLLHRMEMDSDEDDENEEGSDASSDLFDLENFAAGAPDAAAAYRDELPVYETTRVVLGHRAIGHGRSARVV

SEQ ID NO: 12 - DNA, 951 bp, Zea mays
atggagaggtggggcgataaggatcgcggtgcggcagttcccgcaccgggtagacttaggagatacgccgaccagccttcgttctcgagctccctccttgacgcgatctacaagagcatggacgagcccggagatggagctacttcagcagcagctgcaggagccaccaagatgcagagccaccaggaccttcactactcctactactacaagaccagcctggccggttcttataggggctctcgcgctgcagcagctgcacatgcagctaccaccacaagctcctcttcagaatgctcatcctacggtggattctcctcatcagaagcggagtcctcgcagcacagacgcctgagccctatccgcacatcggtcggtgcagctgcttctcccgcaccagcaccagagaagaagaagaaggcgggcgctaacataagagcgaagctcagagatctccgcaagccagcttcaccgggtgcaaggctcgcaggattcctgaacaccatcttctccggaagaagagcacctgcaacaccaccttcgagaggtgcagaatcatcggcctgctccacagcctcttcatactctcggtcctgcctgagcaagacaccttcgactagagggcagcccaagcgcaccgtcaggttcttggactccgacgatggtgaggcagcggctgctgcacctggtggtgagaggagaagagttcaagtgggagtcgcggagctggagaggatgcttctgcacagaatggagatggactccgacgaggatgacgagaacgaggaaggctcggacgcctcttccgatctgttcgacctcgagaactttgcagctggcgctccagatgccgcagcagcctatcgcgatgagttgcctgtgtacgaaaccacacgcgttgtcctcggacacagagctatcggccacggtagaagcgcgcgcgttgtgtag

SEQ ID NO: 13 - protein, 311 aa, Zea mays
MERWRDKDKGAAAPAPGRARRYGDQPSFSSTLLNAIYKSMDEPDDGVTSSSSTAAKKQNQDLRHSCYYYKASLAAGSYRGSSRAAAPRGPQAATTSSSSDQCSSYGGFSSSEAESSQHRRLRPIRTSLATTAVAVAAPALAPEKKKAGVSIRARLRDLRRKPASPGPGARLAGFLNAIFSGRRAPPSASSCSRSCLSETPSTRGQPKRTVRFLDSDGGGERRRTVPVGVAPELEQMLLHRMEVDSGDDDDDESSDASSDLFDLENFAAVDPDGGAAYSDELPVYETTRLVLGHRAIGHGYARTGGVPTRVV

SEQ ID NO: 14 - DNA, 936 bp, Zea mays
atggagaggtggagggacaaggacaagggagcggcggctccggcgccagggagggcgcggcggtacggcgaccagccgtccttctcgtccacgctcctcaacgccatatacaagtccatggacgagcctgacgacggggtaacatcgtcctcctcaaccgccgccaagaagcagaaccaggacctgcgccacagctgctactactacaaggcgtcgctggcggcggggagctaccgcggcagcagcagggcggcggcgccccgggggccacaggccgccaccacgtcgagctcctccgatcagtgctccagctacggcgggttctcgtcgtcggaggcggagtcgtcgcagcaccggcgcctgcggcccatacgcacgagcctcgccaccaccgcggtggccgtggccgcgcccgcactcgcgccggagaagaagaaggccggcgtcagcatccgcgccaggctgagggacctccgccgcaagccggcgtcccccggccccggcgcgcggctggcggggttcctcaacgccatcttcagcggcaggcgcgcgccgccgtcggcgtcctcctgctcgcgctcctgcctcagcgagacgccgtccacgcggggccagccgaagcggaccgtgcggttcctggacagcgacggcggcggcgagcgccggaggacggtgccggtcggggtggcgccggagctggagcagatgctgctccaccggatggaggtggacagcggcgacgacgacgacgacgagagcagcgacgccagctccgatctgttcgacctcgagaatttcgcggccgttgacccggacggcggcgccgcgtacagtgacgagctgccagtgtacgagacgacgaggctggtgctgggccaccgcgccattggccacgggtacgcgcgcacgggaggagtacccaccagagtggtgtga

SEQ ID NO: 15 - protein, 312 aa, Zea mays
MERPWRDKDKGAAAPAAGRARRYGDQPSFSSTLLDAIYRSMDEPDDGVTSSSSTAAKKQNQDLRHSCYYYKASLAAGSYRGSSRAAAPRGPQAATTSSSSDQCSSYGGFSSSEAESSQHRRLRPIRTSLATTAVAVAAPALAPEKKKAGVSIRARLRDLRRKPASPGPGARLAGFLNAIFSGRRAPPSASSCSRSCLSETPSTRGQPKRTVRFLDSDGGGERRRTVPVGVAPELEQMLLHRMEVDSGDDDDDESSDASSDLFDLENFAAVDPDGGAAYSDELPVYETTRLVLGHRAIGHGYARTGGVPTRVV

SEQ ID NO: 16 - DNA, 939 bp, Zea mays
atggagaggccgtggagggacaaggacaagggagcggcggctccggcggcggggagggcgcggcggtacggcgaccagccgtccttctcgtccacgctcctcgacgccatatacaggtccatggacgagcctgacgacggggtaacatcgtcctcctcaaccgccgccaagaagcagaaccaggacctgcgccacagctgctactactacaaggcgtcgctggcggcggggagctaccgcggcagcagcagggcggcggcgccccgggggccacaggccgccaccacgtcgagctcctccgatcagtgctccagctacggcgggttctcgtcgtcggaggcggagtcgtcgcagcaccggcgcctgcggcccatacgcacgagcctcgccaccaccgcggtggccgtggccgcgcccgcactcgcgccggagaagaagaaggccggcgtcagcatccgcgccaggctgagggacctccgccgcaagccggcgtcccccggccccggcgcgcggctggcggggttcctcaacgccatcttcagcggcaggcgcgcgccgccgtcggcgtcctcctgctcgcgctcctgcctcagcgagacgccgtccacgcggggccagccgaagcggaccgtgcggttcctggacagcgacggcggcggcgagcgccggaggacggtgccggtcggggtggcgccggagctggagcagatgctgctccaccggatggaggtggacagcggcgacgacgacgacgacgagagcagcgacgccagctccgatctgttcgacctcgagaatttcgcggccgttgacccggacggcggcgccgcgtacagtgacgagctgccagtgtacgagacgacgaggctggtgctgggccaccgcgccattggccacgggtacgcgcgcacgggaggagtacccaccagagtggtgtga

SEQ ID NO: 17 - protein, 333 aa, Zea mays
MERWAPAPPSAARERPRRRPGQPSFSSTLLDAICDSLDEQAGGHGATAERATAPTPRSAKEQHQAALHYYYYKPFLAASHRAARAAPSPADDCSSGRGYFSSSEVEYSLRRLRPIRTSAGGVGPASVAPVEKQKPAPPGTAKRARKPSAAPASGGCRRPASPGARLASLLNAIFSGKRNSARQHPAPADEEPACSTAPSTARPCLAKTPPSARARARATRNRSRTVRFLDIEGEVAVAAAAAGCRRFPVVEVEDSDGGEESSDASSDLFELENLAALAPANGGPGCRRTCENELPVYGTTGAGLGNDSRLVRRRRPFGYVSHGRSCRGLFDFK

SEQ ID NO: 18 - DNA, 1002 bp, Zea mays
atggagaggtgggcgccagcgccaccgtcggcggcgcgggagaggccgaggcggcgacctggccaaccatccttctcgtccacgctgctggacgccatctgcgattccttggacgagcaggccggcggccacggagcaacggcggagcgtgccacggcaccgacgcctcggagcgccaaggagcaacaccaggcggccctacattactactactacaagcccttcttggccgccagccaccgggcggcgcgcgcggccccttcgcctgcggacgactgctcctccggccgtggctacttctcgtcgtccgaggtcgagtactccctccgccgcctccgccccatccgaacctcagctggcggcgttgggccggcctcggtggctcccgtggagaagcagaagccggccccgccaggcacggcgaagagggcgcggaagccgtccgccgcccccgccagcggcggctgccgcaggccggcctcccccggcgcgcggctcgccagccttctcaacgccatattctccggcaagcggaattctgcgcggcagcacccggctccggcggacgaggagccggcgtgctcgacggcgccatccaccgcgcgcccctgcctcgccaagacaccgccgtccgcgagggcccgggccagggcgacccggaaccgtagcaggaccgtgcggttcttggacatcgaaggcgaggtggctgtggctgcggccgccgctggttgcaggcgattcccggtggtggaagtggaggacagcgacggcggcgaggagagcagcgacgcgagctccgacttgtttgagctagagaacctcgcagcccttgctcccgcgaacggcgggcccggttgccgtaggacgtgcgagaacgagcttccggtgtacgggacgactggagctggtcttgggaacgacagtagacttgtccgtcgtcgacgcccgtttgggtacgtcagccatggtcggagttgcagagggttgtttgatttcaaatag

SEQ ID NO: 19 - protein, 311 aa, Zea mays
MERWAPPSVVREKPRRRPGQPSFSSTLLDAICDSLDEQAGGHGATAAPTRGSAKKQQQAPLHYYYYYKPSLAASNRAARADDCLSGRGYFSSSEVECSLRRLRPIRTSGGAGPASVAPEEKQQLAPPGAAMRARKPFATPASDSGNCRRPASPGARLASLLSAIFSGKRQHPAPADDEPACSTAPSSVRPCLAKTLPSARARVTRRQSRSRTVRFLDIEGDVAVAAAAAGCMRFPVVEVKDSGGSEESSDASSDLFELESLAALAPVNGISGCRRTFENELPVYGDDWNWAWARHCSRPSPPFGYVSHGRS

SEQ ID NO: 20 - DNA, 936 bp, Zea mays
atggagaggtgggcgccgccgtcggtggtgcgggaaaagccgaggaggcggcctggccagccgtccttctcgtcgacgctgctggacgccatctgcgactccttggacgagcaggccggcggccatggagcgacggcagcaccaacgcgtgggagcgccaagaagcagcaacaggcgcccctgcattactactactactacaagccctccttggcggccagcaaccgcgcggcgcgcgcggacgactgcttgtccggccgtgggtacttctcgtcgtccgaggtcgagtgctccctccgccgcctccgccccatccgcacctctggcggcgccgggccggcctcggtggctcccgaggagaagcaacagctggcaccgcctggtgcagcgatgagggcgcggaagccgttcgccacccccgccagcgacagcggcaactgccgcaggccggcctctcccggcgcgcggctcgccagtttgctcagcgcaatcttctccggcaagcggcagcacccggctccggcggacgacgagccggcgtgctcgacggctccatccagcgtgcgcccctgcctcgctaagacactgccgtcggcgcgggcgcgggtgacccggaggcagagccggagcaggaccgtgcgtttcttggacatcgaaggcgatgttgctgtggccgcagcagccgccggttgcatgagattcccggtggtcgaagtgaaggacagcggtggcagcgaggagagcagcgacgcaagctccgacctgttcgaactggagagcctcgcagccctcgctccggtgaacggcatctccggttgccgtagaactttcgaaaacgagcttccggtgtacggcgacgattggaactgggcttgggcacgacattgcagtcgtccgtcgccgccgtttgggtacgtcagccacggtcggagttag

SEQ ID NO: 21 - protein, 350 aa, Zea mays
MERRGNCHGGKHPPPRQARGPGERTRQPSSGGSFSASLLDAVYRSLDDGDGADVVVDAARGSGAEEKAAATAQFWWANKEAATARPRQSSSSADGDRRRRETGAARPRHSGYASSTTSSSDSSASYSSFSCSSASTTDTESTCRRHSPPPPRMSLSEESAATDAEEATATPPPPPKSKPKKKARPCFPVARIRPPKASVPSSSSSGAQPPSPATFACALKALFSSVRLQRKPKAPAATPPPKISQPQPQPQPPSMSATSTAKAADAPAEPSVQRTVRLRPEAEVSVVRRRVVEELVRSLEELEADEEGSDASSDLFELDSLRGAGADELPVYGTTSLVVANRAIAQGPAR

SEQ ID NO: 22 - DNA, 1053 bp, Zea mays
atggagcgccgcggaaattgccacggcggcaagcacccgccgccgcgccaggcgcgagggcccggggagcgcacccggcagccgtccagcggcggctccttctcggcatcgctcttggacgccgtctaccgctccctcgacgacggcgatggtgcggacgtcgtcgttgacgccgcgcgtgggagtggtgcggaggagaaggcggccgcgacggcgcagttctggtgggcgaataaggaggcggccacggccagacccaggcagtcctcgtcgagcgcggacggggacaggcgacgccgggagacgggagctgcgcgcccgcgccactcggggtacgcgtcgtcgaccacgtcgtcgtccgactcgtcggctagctacagcagcttctcctgctcgtcggcgtcgaccacagacactgagtccacgtgccgccgccacagccccccgccgccgcggatgtcattgtcagaggaatccgccgccacagacgccgaggaggcaaccgcaacccctccaccgccacccaagagcaaaccgaagaagaaggccaggccgtgtttccccgtagcaagaatccgaccaccaaaggcctcggtgccatcatcatcgtcgtctggagcgcagccgccatcgccggcgacgttcgcgtgcgcccttaaggctctgttctcctcggtgcgcctccagaggaagcccaaggctccggctgcaacccctccgcccaaaatctcgcagccgcagccgcagccacagcctccgagcatgtcggcgacgagcactgcgaaggcggctgacgcgccggcggaaccgtcggtgcagaggacggtgaggctccgcccggaagccgaggtgtcggtggtgcggagaagggtggtggaggagctggtgcggagcctcgaggagctggaggcggacgaggaggggagcgacgccagctccgacctcttcgagctggacagccttcgtggcgccggcgcggacgagctgcccgtgtacggcacgaccagcctcgtggtggccaaccgcgcgatcgcccagggaccagctcgttga

SEQ ID NO: 23 - protein, 347 aa, Zea mays
MERRGHCHGGKPPHPHPPPRRVRTRQPSSGSFSASLLDAIYRSLDDGDGADVVVDAARGSVEEKAAATATAQFWWLNKAAAPKPCRQSSSTADRDRRRREAGVARPRHSGSGYASSTASSSDSSAASYSSLSCSSASTVGIESTCRRHGLPPPRVSLSEESVATDAEETTPPPPNSKPKKKARPCFPVARIRPRASVPPPSSGPQPPSSPATFACALKALFSSARLQRKPKAPAPARTTPPPKISHPPRMSTTSAAKAADAPQPSEPTTVRLHPEAEASVVRRRVEELVRGLEELEEDEERSDASSDLFELESLRGAGADELPVYGTTSLVANRAIAQGPGGQLVNK

SEQ ID NO: 24 - DNA, 1044 bp, Zea mays
atggagcggcgcggccattgccacggcggcaagcccccgcacccgcacccgccgccgcgccgggtgcgcacccggcagccgtccagcggctccttctcggcgtcgctcctggacgccatctaccgctccctcgacgacggcgacggcgccgacgtcgtcgttgacgccgcgcgtgggagtgtagaggagaaggcggccgcgaccgcgacggcgcagttctggtggttgaacaaggcggcggcgcccaagccctgcaggcaatcgtcgtcgaccgcggacagggacaggcgccggcgggaggcgggagttgcgcgcccgcgccactcggggtcggggtacgcgtcgtcgaccgcgtcgtcgtccgactcatcggctgctagctacagcagcctctcctgctcgtcggcgtcgaccgtaggtatcgagtctacgtgccgccgccacggcctgcccccgccgcgggtgtcattgtcagaggaatccgtcgccacggacgcggaggagacgaccccgccgccacccaacagcaaaccaaagaagaaagccaggccatgtttccccgtggcaagaatccgaccaagggcctcagtgccaccaccatcgtccgggccacagccgccgtcgtcgccggcgacgttcgcgtgcgcactcaaggctctgttctcctcggcgcgcctccagaggaagcccaaggctccggcaccagcaagaacgacccctccgcccaaaatctcgcatcctccgcgcatgtcgaccacgagcgctgcgaaggctgctgatgcgccgcagccgtcggagccgacgacggtacggctccaccctgaagccgaggcgtcggtggtgcgacgaagggtggaggagctggtgcggggcctcgaggagctggaggaggacgaggagaggagcgacgccagttccgacctcttcgagctggagagccttcgtggcgccggcgccgacgagctgcccgtgtacggcaccaccagcctcgtggccaaccgcgctatcgcgcaggggccaggcggccagctcgttaataagtaa

SEQ ID NO: 25 - protein, 324 aa, Zea mays
MPPHDGARAPPPRPSRGHQPSFSAALLDAIYHSLEADAEARTSTEARRTRTPASSPAQLPSRRRPTPELSPSPSPSRSSVRSPRLQRAPRPCRVRPDPQPNSSGSLLLPPPLPPHPHEHEPSSTGHRRVADAERKRGRGRKSKRTAPFACLLNALLCNRRPARPVDRTPRATATPTPTPATAAPEPASARSILSSRASRSRRESAAAGGVLAPARRAVRFSPVATVVGDGHGHGAGSVGTATTGLRAKESAAEAERRVEELLRALGVADERERAKESTESSSDLFELDSLPAFQDRGTDLPRSRTVADGDDGAGLLARPRPRVQ

SEQ ID NO: 26 - DNA, 975 bp, Zea mays
atgcctccgcacgacggcgcccgagccccgccgccccggcccagccggggccaccagccgtccttctccgccgcgctcctcgacgccatctaccactccctcgaggccgacgccgaggcgcgcacgtccacagaggcgcgccggacgcgcacgccggcgtcgtccccggcgcagcttccgagccgacgtcgcccgacgcccgagctgtcgccgtcgccgtccccgtcccgctcctcggtccggtcaccgcggctgcagagggcgccgcggccgtgccgcgtccggccggacccgcagcccaactccagcggctccctcctcctgcctccgccccttccgccgcacccacacgaacacgagccgtcgtcgaccgggcaccgccgcgtcgcggacgcggagaggaagaggggccgcgggaggaagagcaagaggacggcgccgttcgcgtgcctcctcaacgccctgctctgcaacaggaggccggcgaggccggtcgaccgcacgccgcgtgcaacggcaacgccaacgccaacgcccgccaccgcggcgccggagccggcgtcggcgaggtcgatcctctcgtcgcgcgcctcgcggagccggagggagtccgcggcggcgggaggggttctggccccggcgaggcgggcggtgcggttctcgcccgtggcgacggtagtgggcgacgggcacgggcacggcgctggatcggtcgggacggcgacgacggggctgcgggccaaggagtccgcggcggaggcggagaggagggtggaggagctgctgcgcgcgctcggcgtcgcggatgagcgggagagggctaaggagagcaccgagtccagctccgacctgttcgagctcgacagcctgcccgcattccaagacagaggcaccgacctgccgcgttccagaaccgtcgccgacggcgacgatggcgccgggctgctggcacggccgcggccccgcgtgcagtga

SEQ ID NO: 27 - protein, 340 aa, Ananas comosus
MERWKKPPPPRARRGYSCDHPSFSSTLLDAIYRSIDEPEGAGAAAKPPPPPAAAWRSATVRERDAAPRRPQQQPQPPQQQRRCGAWPTYSTSSSSECSSYGGFSSSEAESSAPGSARLKPIRTGSRPGPVHHHPAVQSPGLAQPLPEKKKSSSIRSRIREFRRSSKAPASPGARLASFLNSIFAAAAGNPRKPSKRPASAAAASCGESASACSTASSYSRSCLTKTPSTRGGLPPAAPPPPPPKRSVRFGPVSVIVDEELRPYEKKLPYDGDPLPMARKRAEEMALGFHREEEEEEEEGESDSSSDLFELESLTVTGDELPVYETTSFLTNRAIAHGLIV

SEQ ID NO: 28 - DNA, 1023 bp, Ananas comosus
atggagaggtggaagaagccgccgccgcctcgggcgcggcgagggtatagctgcgaccacccctccttctcctccacactcctcgacgccatctaccgctccatcgacgagcccgagggcgccggcgcggcggcgaagcccccgccgccgcccgccgccgcgtggaggagcgccaccgtgcgggagcgggacgcggcgccgcgccgcccccagcagcaaccgcagccgccgcagcagcagcgccgctgcggcgcgtggccgacgtactcgacctcgagctcgtccgagtgctccagctacggcgggttctcgtcgtccgaggccgagtcgtcggcgcccggctcggcccggctcaagccgatccggaccgggtcccggcccggcccggtccaccaccaccccgcggtccagtcccccgggctcgcgcaacccctcccggagaagaagaaatcgagctcgatccggagccggatccgggagttccgcagatcatcgaaggcccccgcttccccgggggcgcgcctcgcgagcttcctcaactcgatcttcgccgccgcggccggaaaccctaggaagccctccaagcgccccgcctccgccgccgccgcgagctgcggcgaatcggcctcggcgtgctccacggcgtcgtcctactcgcgctcgtgcctcaccaagactccatcgacccgcggcgggctcccccccgcggcgccgcccccgccgccgccgaagagatctgtgcggttcggccccgtgagcgtgatcgtcgacgaggagctccgcccctacgagaagaagctcccgtacgacggcgatcctctaccaatggcgaggaagagagcggaggagatggcgctagggtttcatagggaggaggaggaagaggaggaggaaggggagagcgattcgagctcggatctcttcgaattggagagcttaactgtgacgggtgacgagctcccggtgtacgagaccaccagcttcctcaccaatcgcgccatcgcccatggtctcatcgtgtaa

SEQ ID NO: 29 - protein, 357 aa, Arabidopsis thaliana
MEITWEKPKSSSHHRNPSFSSTLLDQIYRSIDDSSPPPPLESIKKKKHHHQQRNASLHEDREISPIYHRRSIAADFERSRRKTDFLRHSNSSSSDSSGFSSSESDSFHGRSKSSASPPSSSRQQPKPIRTSSVDHSSAVQKPKELGGFLRTKSKALKIYSDLKKVKQPISPGGRLATFLNSLFTNAATNPKKHKKTTTVAVVEEPHSSSTCSSASSFSRSCLSKTPSSSGKSKRSVRFCPVNVILDEDSSFTMPYAYNNERLYDNNEAKRVEEHRRVIQAAKDLLRTYHNKNKVTTTNINNVEEDDEDDAASCASSDLFELENLSAIGIERYREELPVYETTRLDNMNRVIATGLIV

SEQ ID NO: 30 - DNA, 1074 bp, Arabidopsis thaliana
atggagatcacttgggagaaacctaaatcttcatctcaccaccgtaacccttctttctcctccactctcctcgaccaaatctaccgctccatcgacgattcctccccccctcctcccctcgaatccatcaagaagaagaagcatcaccaccaacaacgtaacgcttctctacacgaagatcgtgaaatctctcctatctatcaccgccgctcaatcgccgctgatttcgagagatctaggaggaagacagactttctcagacattccaactctagctcttctgattcaagcggattctcctcttccgagtcagattcattccacggacgctccaaatcttccgcttctcctccttcttcttctcgtcaacaaccgaaaccgattcgaacatcatccgtcgaccactcctccgccgtccagaaacctaaagaactcggtggcttcttgagaacgaaatcaaaagcgttaaagatctactccgacttgaaaaaagtcaaacaacctatctctccgggcggcagactcgctactttccttaactctcttttcaccaacgccgccactaaccctaagaagcacaagaaaactaccaccgtcgccgtcgtggaagaaccacactcctcctccacatgctcctccgcgtcgtccttctctaggtcttgcttgagcaaaactccatcgtctagcggcaaatcaaaaagatccgtacggttctgtccagtcaatgtgatccttgacgaagattcctcctttaccatgccttacgcttataacaacgaaagactctacgataacaacgaagctaaacgcgtggaagagcatcgccgtgtcatccaagcagccaaagatcttcttagaacttaccataacaagaacaaagttacaactacgaacatcaataacgtagaagaagatgatgaagacgatgctgcgagttgtgcaagctctgatctattcgagttggagaatctgtcggctattgggatcgagaggtatagagaagagcttccggtttacgaaaccactcgtcttgataatatgaatcgagtcattgctactggtttaattgtataa

SEQ ID NO: 31 - protein, 366 aa, Arabidopsis thaliana
MDPWDTYNIPLDHQHRLRNRRDHRHPSFSSTLLDQIYRSIDDSSTNSSSMRKTKHQNREDTRVSANRRDDFNRSKNLKTIEPVFFKHSSSSSSDSSGFSSSESDYFYRRSKSSPAISHPKPIRTTVERFERSPQNHRPNSSNKQEHGSFLKTKSKALKIYSDLKKVKQPISPGGRLATFLNSIFTGAGNTKKLNKINTTVTSTTAAAAAASSTTTCSSASSFSRSCLSKTPSSSEKSKRSVRFCPVNVIFDEDSSKYNNKNNKVYGNNEREYESIRHTLENRVMEENRRVIEAAKELLRSYQKKNKEVIEVSVEDDEEDDDDDALSCTSSDLFELDNLSAIGIDRYREELPVYETTRLNTNRIISR

SEQ ID NO: 32 - DNA, 1101 bp, Arabidopsis thaliana
atggatccttgggatacttataacataccactagaccaccaacaccgtctccgtaaccgccgtgaccaccgtcatccttccttctcctctactctcctcgaccaaatttaccgctccatcgacgattcctctactaactcctcatccatgagaaaaaccaaacatcagaaccgcgaggacacgcgtgtttctgctaaccgccgcgacgatttcaacagatccaagaatcttaaaaccatagaacctgttttcttcaaacattcaagctcaagctcctctgactcaagcggattctcgtcttccgagtctgattatttctacagacgatctaaatcctcgccggcgatttctcatcctaaaccgatacgaaccaccgtggaacgattcgaaagatctcctcagaatcaccgacctaacagtagtaataaacaagaacatggaagctttctcaaaacgaaatctaaagcgttgaagatttatagcgatttgaagaaagtgaaacaaccgatttctcccggtggtcgtctcgctactttccttaactccatattcaccggcgccggaaacactaaaaaacttaacaaaatcaacacaaccgtcacttccaccaccgccgccgccgccgcagcgtcatcaaccactacatgctcctctgcttcatcattctccagatcttgcttgagcaaaacgccatcgtctagcgaaaaatcgaaaagatccgtacgtttttgtcccgtcaacgtcatcttcgacgaagactcctccaagtacaacaacaagaacaacaaagtgtacggaaacaacgaacgtgaatatgaatccattcgtcatactctagagaaccgagtcatggaagagaatcgtcgtgtaatcgaagcagctaaagaacttctgagaagttaccagaagaagaataaagaagtcattgaagtctccgtcgaagacgatgaagaagacgacgatgatgatgcgttgagttgcacaagctctgatctgttcgagttggataatttgtcggcgataggaatcgataggtatagagaggagcttccggtttacgaaacaactcgcttgaacactaatcgtatcatttctagatga

SEQ ID NO: 33 - protein, 327 aa, Brachypodium distachyon
MERRGEKGAAAARARRPQGAAEQPSFSSTLLDAIYKSMDEPGHDAVSKKKQQEKEEALHYSYYYRPSLAGSYRARAPGPAHATTTTSSSSDCSSYGGFSSSEAETSSGRHHHRRLRPIRTAAAAPAPPAPEKKASKKQQVAPGASIRAKLRDLRKAPASPGARLAGFLNTIFAGGGGKRAPQTPPSASAAAEYACSTASSAASYSRSCLSKTPSTRGGGGQQQAGRTVRFVDSAAEAPATVPGRRMPARAVEQMLLRRMEMESDEEDEESSDASSDLFELENFTAAPPGAAGYRDELPVYETTRVVLNRGSIGGHHGYGHGRSARVV

SEQ ID NO: 34 - DNA, 984 bp, Brachypodium distachyon
atggagaggcggggtgagaagggggcagcggcggcgagggcgaggcggccgcagggcgccgccgagcagccgtccttctcgtcgacgctgctggatgcgatatacaagtccatggacgagccgggacatgacgctgtcagcaagaagaagcagcaggagaaggaggaagccctgcactacagctactactacaggccgtcgctggcggggagctaccgtgcgcgcgcgccggggccggcgcacgccacgacgacgacgtcgagctcgtccgactgctccagctacggcggcttctcctcgtccgaggccgagacatcgtccggccgccaccaccaccgccgcctgcgccccatccgcacggccgccgccgcccccgcgccaccggcgccggagaagaaggcgagcaagaagcagcaggtggcgccgggcgcctcgatccgcgccaagctgagagacctccgcaaggcgccggcgtcccccggcgcgcggctcgcgggcttcctcaacaccatcttcgccgggggcggcggcaagcgcgcgccgcagacaccgccttccgcgtccgcggccgcggagtacgcgtgctctactgcatcctcggcggcgtcgtactcgcgctcgtgcctgagcaagacgccctcgacgcgcggcggcggggggcagcagcaggcgggccggacggtgcggttcgtggacagcgccgccgaggcgccggccacggtgcccggccggaggatgccggcgcgggcggtggagcagatgctgctccgaaggatggagatggagagcgacgaggaggacgaggagagcagcgacgccagctctgacttgttcgagctcgagaacttcactgctgctccgccgggcgcggcagggtacagggacgagctgccggtctacgagacgacccgggtcgtgctcaaccgtggaagcatcggcggccaccatggctacggccacggccggagcgccagagttgtctga

SEQ ID NO: 35 - protein, 318 aa, Hordeum vulgare
MERRGMEKGTSAGARRHADQPSFSSSLLDAIYRSMEDEPGHAGGEAAVTKKQQQQKKHVEEALHYSYYYRPSLAGSYRAPGPHATTSSSSECSSYGGFSSSEAESSNHRRLRPIRTVVPGGQPAPAPEKKAKKPAAAGASIRAKLRDLRKPASPGARLAGFLNTIFAGKRSAPQTPPSARAAAAAEYACSSASSYSRSCLSKTPSTRGHANRTVRFADGDREAPATVPGADRRRVPALAVEQMLLRRMEMESDEDEDDDEDSSDASSDLFELENFAAAVAVPPGAGYRDELPVYETTRVVLSRGIGHAYGHGRSARVV

SEQ ID NO: 36 - DNA, 957 bp, Hordeum vulgare
atggagaggcgtggcatggagaaggggacgtcggcgggggcgaggcggcacgccgaccagccgtccttctcgtcgtcgctgctcgacgcgatatacaggtccatggaggacgagccgggccacgccggcggggaggcggcggtgaccaagaagcagcagcagcagaagaagcacgtggaggaggccctgcactacagctactactacaggccgtcgctcgccgggagctaccgggcgccggggccgcacgccaccacgtccagctcctccgagtgctccagctacggcggcttctcctcgtccgaggccgagtcgtccaaccaccgccgcctgcgccccatccgcacggtggtgcccggcggtcagcccgcgcccgcgccggagaagaaggccaagaagccggcggcggcgggggcctcgatccgcgccaagctcagggacctccgcaagccggcctcccccggcgcgcgcctcgcgggcttcctcaacaccatcttcgccggcaagcgcagcgcgccgcagaccccgccctcggcccgggcggcggcggcggcggagtacgcgtgctcctcggcgtcgtcctactcgcggtcgtgcctgagcaagacgccgtccacgcgcgggcacgcgaaccgcaccgtgcggttcgcggacggcgaccgcgaggcgcccgccacggtgcccggggcggaccggcggagggtgccggcgctggcggtggagcagatgctgctccggcgcatggagatggagagcgacgaggacgaggacgacgacgaggacagcagcgacgccagctccgacctgttcgagctggagaacttcgccgccgccgtcgccgtgccgccgggtgcggggtacagggacgagctgccggtgtacgagacgaccagggtggtgctgagccgcggcattggccacgcgtacgggcacggccggagcgccagggtggtctga

SEQ ID NO: 37 - protein, 337 aa, Oryza sativa
MRDMEMRWAAPAPAARGRGRARRRAPDQPSFSSTLLDAICDSMDEGGEDGRTRNAASAAAKKRQEAANSYHYYYCYKPSLAASYRAAPALGSTADCPGRGYFSSSEVEYSLRRLRPIRTSAAGGAGDGAAVARKQRHEQPDVEKTAKTKPGSASARACRRPASPGARLASLLNSIFSGKRPSAQRPACSPDYPEPACSTAPPSSSSSYARRPCHAKTPRTPPTTTTTARARPSRSRTVRFLDIDGKVAVAAAVAGCRRIPVMEVEADTDDGGEESSDASSDLFELDSLAAIAPAGGRDGSYGDELPVYGTTGVGIRRDIGRRRPYGHAPCRSWSRAV

SEQ ID NO: 38 - DNA, 1014 bp, Oryza sativa
atgcgcgacatggagatgaggtgggcggcgccggcgccggcggcgagggggagggggagggcgaggcggcgggcgcctgaccagccgtcgttctcgtcgacgctgctcgacgccatctgcgactccatggacgagggcggcgaggacggccggacaagaaacgcggcgagtgcggcggccaagaagaggcaggaggcggcgaacagctaccactactactactgctacaagccgtcgctggccgccagctacagggcggcgccggcgctgggttccacggcggattgccccgggaggggctacttctcgtcgtccgaggtggagtactcgcttcgccggctccgccccatccgcacctccgccgccggtggcgcgggagacggcgcggcggtcgcgcggaagcagcggcatgagcagccggatgtggagaagacggcgaagacgaagccgggctccgcctcggcccgcgcgtgccgcaggccggcgtcgcccggcgcgcggctcgccagcttgctcaactccatcttctccggcaagcgtccatccgcgcagcgcccggcgtgttcgccggactacccggagcccgcgtgctcgacggcgccaccgtcgtcgtcgtcgtcgtacgcgcgccgcccctgccacgccaagacgccgcgcaccccccccaccaccaccaccacggcgagggcgcggccgagccggagcaggaccgtccggttcctggacatcgacggcaaggtcgcggtggccgcggccgtcgccggctgccgccgaattccggtcatggaggtggaggccgacaccgacgacggaggtgaggagagcagcgacgcgagctcggacctgttcgagctcgacagcctcgcggccattgctccggcgggtggtcgcgacggctcctacggggacgagctgccggtgtacgggaccaccggagttgggatccgccgcgacattggccgccgccgtccgtacggccatgctccttgtcggagctggagtagggctgtctag

SEQ ID NO: 39 - protein, 310 aa, Oryza sativa
MERWAAPKVTAGSARRYVADQPSFSSTLLDAIYKSMDEQPGHGGGATGVEAVAAAAKKQHEAALHYGNYYKPSLAGSYRARAPGPHATTSSSSECSSYGGFSSSEAESSHHRRLRPIRTTVPGGAPGPAPEKKAKKPGASIRAKLRDLRKPASPGARLAGFLNSIFAGKRAPATPPSATAGAESACSTASSYSRSCLSKTPSTRGQAKRTVRFLDSDTESLASSTVVDRRRVPVEAVQQMLLQRMEMESDEDDDESSDASSDLFELENFAAIAPAGAAYRDELPVYETTRVALNRAIGHGYGHGRSARVV

SEQ ID NO: 40 - DNA, 933 bp, Oryza sativa
atggagaggtgggcggcgcccaaggtgacggctggttcggcgaggcggtacgtcgccgaccagccgtctttctcatccacgctgctcgacgcgatatacaagtcgatggacgagcagccgggtcacggcggcggcgccaccggggtggaggcggtggctgcggcggccaagaagcagcacgaggcggccctgcactatgggaactactacaagccgtcgctcgcggggagctaccgggcgcgcgcgccgggtccgcacgccacgacgtcgagctcgtcggagtgttctagctacggcgggttctcgtcgtccgaggcggagtcgtcgcaccaccgacgcctccgccccatccgcacgactgtccccggtggtgcgccggggcccgcgccggagaagaaggccaagaagcccggggcctccatacgcgccaagctcagggacctccgcaagccggcttcccccggcgcgcgcctcgcgggcttcctcaactccatcttcgccggcaagcgcgcgccggccacgccgccctcggcgacggccggggcggagtccgcgtgctcgacggcgtcgtcctactcccgctcctgcctgagcaagacgccgtcgacgcgcgggcaggcgaagcggaccgtgcggttcttggacagcgacacggagtccctggcgtcgtcgacggtggtcgaccgccgcagggtgcccgtggaggcggtgcagcagatgctgctccagcggatggagatggagagcgacgaggacgacgacgagagcagcgacgcgagctccgacctgttcgagctcgagaacttcgccgccattgctcccgccggggccgcgtaccgggacgagctgccggtgtacgagacgaccagagtggcgctgaaccgcgccattggccatgggtatggccatggacggagcgccagagttgtctga

SEQ ID NO: 41 - protein, 368 aa, Phoenix dactylifera
MERWAKPPVRRGNDNPSFSSTLLDAIYRSIDEADGGAAREHFTGGAPDRRDASVPAAAKKQQSFFTDEWPAEGRRTAVVSEKAVTRLRKPENRPRFPVNSTSSSSDCSSYGGFSSSEAESVPRPSGLRPIRTGGLLYRSERARSNPAPPQPPAVGSPSAHHHQEKKKSGSIRSKFRDLRKAKAPASPGARLASFLNSLFTANGNSRKSKSAAAAYGGSGVRGGEESACSTATSYSRSCLSKTPSSKGRPAVAAAAGQGVKRSVRFYPVSVIVDEDCRPCGQKCLYEGDSAAVARRPPEAATIAKKRVEELLRGFEEEEEDEEASDSSSDLFELENLTAIGGRGYRDELPVYETTHLGTNRAISHGFFL

SEQ ID NO: 42 - DNA, 1107 bp, Phoenix dactylifera
atggagaggtgggcgaagccgccggtgcggcgcggcaacgataacccgtccttctcctccactctcctcgacgccatctaccgctccatcgacgaggccgacggcggagccgcgagggagcacttcaccggcggagcacccgaccgccgagacgcctccgttcccgcggcagcaaagaagcagcagagctttttcaccgacgagtggccggcggaagggaggaggaccgcggtggtgagcgaaaaggcggtgacacgacttcgcaagcccgaaaaccgccccaggttcccggtgaactcgacctccagctcctccgactgctccagttacggcgggttctcgtcctccgaggcagagtcggtgccccgcccttccgggctccggccgatccggaccggcggtcttctttaccggtctgagagggcccggtcgaaccccgcgcctccccagccgccagctgtagggtcgccctcggctcaccaccaccaggagaagaagaagtcgggctcgatccggagcaagttccgggatctaaggaaggccaaggcgccggcatcccctggagcccgcctcgccagcttcctcaactccctcttcaccgccaacggcaactcgaggaagtccaaatccgccgctgccgcttacggcggcagcggcgtgagaggaggggaggagtcggcgtgttcgacggcaacatcgtactcgcggtcgtgcctgagcaagacgccgtcgtcgaaggggcggccggcggtggcggcggcggccggccagggtgtgaagaggtctgtgaggttctacccggtgagcgtgatcgtggacgaggactgccggccgtgcgggcagaagtgcctgtacgaaggggattcggcggcggtggcgcggcggccgcctgaggcagcgacgattgcgaagaagagggtggaggagcttctccgagggtttgaggaagaggaggaggacgaggaggcgagcgattcgagctcagatctgttcgagttggagaacctgacggcgatcggagggcgaggatacagggatgaacttccggtgtacgagaccactcatcttggcactaatcgcgccatttcccacggtttcttcttgtaa

SEQ ID NO: 43 - protein, 346 aa, Sorghum bicolor
MERWGDKDKRAAGAAPGRARRYADQPSFSSTLLDAIYKSMDEPDDGVTSSGAATAAATKKQNHDLHYSYYYKASLAGSYRGSSRAAAPGPHAATTSSSSECSSYGGFSSSEAESSQHRRLRPIRTSAAAGAAATAPAPALAPEQKKKAAKKAGANIRAKLRELRKPASPGASPGARLAGFLNAIFNGRRAPQTPPSASASRGAAAESACSTASSYSRSCLSKTPSTRGQPKRTVRFLDSDDGGEAAAPAPGVERRRVQVGVAELEQMLLHRMEMDSDEEDDEEDEESSDASSDLFDLENFAAVAPDAGAAAAAYRDELPVYETTRVVLGHRAIGHGYAHGRSTRVV

SEQ ID NO: 44 - DNA, 1041 bp, Sorghum bicolor
atggagaggtggggggacaaggacaagcgggcggcgggtgcggcgccggggagggcgaggcggtacgccgaccagccgtccttctcgtccacgctgctcgacgccatatacaagtccatggacgagcccgacgatggggtgacatcatccggcgccgccaccgcggcagcgaccaagaagcagaaccatgacctgcactacagctactactacaaggcgtcgctggcggggagctaccgcggcagcagcagggcggcggcccctgggccgcacgccgccaccacgtcgagctcctccgagtgctccagctacggtgggttctcgtcgtccgaggcggagtcgtcgcagcaccggcgcctgcggcccatacgcacgagcgccgccgccggggcggcggccaccgcgcccgcgcccgcgctggcgccggagcagaagaagaaggccgccaagaaggccggcgccaacatccgcgccaagctgagggagctccgcaagccggcgtctcccggcgcttccccaggcgcgcggctggcggggttcctcaacgccatcttcaacggcaggcgcgcgccgcagacgccgccgtcggcgtcggcgtcgcggggcgcggcggcggagtccgcgtgctccacggcgtcgtcctactcgcgctcctgcctcagcaagacgccgtccacgcggggccagccgaagcggaccgtgcggttcctggacagcgacgacggcggcgaggcggcggcgcccgccccgggcgtcgagcgccggagggtgcaggtcggggtggcggagctggagcagatgctgctccaccggatggagatggacagcgacgaggaggacgacgaggaggacgaggagagcagcgacgccagctccgacctgttcgacctcgagaatttcgcggccgttgcccccgacgccggcgccgccgccgcggcgtacagggacgagctgccagtgtacgagacgacgagggtggtgctgggccaccgcgccattggccacgggtacgcgcacgggaggagtaccagagtcgtgtga

SEQ ID NO: 45 - protein, 327 aa, Setaria italica
MERWGEKGAAPAPGRARRYADQPSFSSTLLDAIYKSMDEPDAAAAATTKKQSQDLHYSYYYKASLAGSYRAGRAASAVATPGPHATTSSSSECSSYGGFSSSEAESSQHRRLRPIRTSVAAAGEAPAPAPEKTKKAAKNKPGANIRAKLRDLRKPASPGARLAGFLNAIFNGKRAPPTPPSASRAAAASESACSSASSYSRSCLSKTPSTRGQPKRTVRFMDSDTEAAAAVPAAGTERRRVQVGVVELERMLLHRMEMDSDEDDESSDASSDLFELENFAAVAPAAGGAGAYRDELPVYETTRVVLNRAIGHGHGHGYAHGRSTRVV

SEQ ID NO: 46 - DNA, 984 bp, Setaria italica
atggagaggtggggggaaaagggggcggctccggcgccggggagggcgaggcggtacgccgaccagccgtccttctcgtccacgctgctcgacgccatatacaagtccatggatgagcccgacgccgccgccgccgccaccaccaagaagcagagccaggacctgcactacagctactactacaaggcgtcgctggcggggagctaccgcgccggaagggcggcgtcggcggtggcgacgccggggccgcacgccaccacgtcgagctcatccgagtgctccagctacggcgggttctcctcgtccgaggcggagtcgtcgcagcaccgccgcctgcggcccatccgcacgagcgttgccgccgccggggaggcgcccgcgcccgcgccggagaagacgaagaaggccgccaagaacaagccgggcgccaacatccgcgccaagctcagggacctccgcaagccggcgtcccccggcgcgcggctcgcggggttcctcaacgccatcttcaacggcaagcgcgcgccgccgacgccgccctcggcgtcgcgcgcggcggcggcgtcggagtccgcgtgctcgtcggcgtcctcgtactcgcgctcctgcctcagcaagacgccgtccacgcgggggcagccgaagcgcaccgtgcgattcatggacagcgacaccgaggcggcggccgcggtgccggcggcgggcaccgagcgccggcgggtgcaggtcggggtggtggagctggagcggatgctgctccaccggatggagatggacagcgacgaggacgacgagagcagcgacgccagctccgacctgttcgagctcgagaacttcgcggccgtcgcgccggccgctggcggggccggcgcgtacagggacgagctgccggtgtacgagacgacgagggtcgtgctgaaccgcgccattgggcacgggcacgggcacgggtacgcgcacgggaggagtaccagagtggtgtaa

SEQ ID NO: 47 - protein, 309 aa, Triticum aestivum
MERRGDKGMAARARRHADQPSFSSSLLDAIYRSMEDEPGHAAGDAAATKKKQQQQEEALHYSYYYRPSLAGSYRARAPAPGPHATTSSSSECSSYGGFSSSEAESSNHRRLRPIRTAVPGEQPAPAPEKKAKKQPAGASIRAKLRDLRKPASPGARLAGFLNTIFAGKRAPQTPPSARTAAEYACSSASSYSRSCLSKTPSTRGHANRTVRFVDSDREAPATVPGADRRRVPVEQMLLRRMEMESDEEDDEDESSDASSDLFELENFAAAAVPPGAGYRDELPVYETTRVVLNRGIGHAHGHGRSARVV

SEQ ID NO: 48 - DNA, 930 bp, Triticum aestivum
atggagaggcgcggggacaaggggatggcggcgcgggcgaggcgccacgccgaccagccgtccttttcgtcgtcgctgctcgacgcgatatacaggtccatggaggacgagccgggccacgccgccggggacgcggcggcgaccaagaagaagcagcagcagcaggaggaggccctgcactacagctactactacaggccgtcgctggccgggagctaccgtgcgcgcgcgccggccccggggccgcacgccaccacgtccagctcctccgagtgctccagctacggcggcttctcctcgtcggaggccgagtcgtccaaccaccgccgcctgcgccccatccgcaccgccgtgcccggcgagcagcccgcgcccgcgccggagaagaaggccaagaagcagccggcgggggcctccatccgcgccaagctcagggacctccgcaagccggcctcccccggcgcgcgcctcgccggcttcctcaacaccatcttcgccggcaagcgcgcgccgcagacgccgccctcggcccgcacggcggcggagtacgcgtgctcgtccgcgtcgtcctactcgcggtcgtgcctgagcaagacgccgtcgacgcgcgggcacgccaaccgcaccgtgcggttcgtggacagcgaccgcgaggcgccggcgacggtgcccggggcggaccggcggagggtgccggtggagcagatgctgctccggcgcatggagatggagagcgacgaggaggacgacgaggacgagagcagcgacgccagctccgacctgttcgagctcgagaacttcgccgccgccgccgtgccgccgggcgcggggtaccgggacgagctgccggtgtacgagacgacgagggtggtgctgaaccgcggcattgggcacgcgcacgggcacggccggagcgccagagttgtctga

SEQ ID NO: 49 - protein, 311 aa, Triticum aestivum
MERRGDKGMAARARRHVDQPSFSSSLLDAIYRSMEDEPGHAAGDAAATKKKQQQQQQEEALHYSYYYRPSLAGSYRARAPAPGPHATTSSSSECSSYGGFSSSEAESSNHRRLRPIRTTVPDGQPAPAPEKKAKKQPAGASIRAKLRDLRKPASPGARLAGFLNTIFAGKRAPQTPPSARTAAEYACSSASSYSRSCLSKTPSTRGHANRTVRFVDSDREAPATVPGADRRRVPVEQMLLRRMEMESDEEDDEESSDASSDLFELENFAAAAVPPGAGYRDELPVYETTRVVLNRGIGHAQHGHGRSARVV

SEQ ID NO: 50 - DNA, 936 bp, Triticum aestivum
atggagaggcgcggggacaaggggatggcggcgcgggcgaggcgccacgtcgaccagccgtccttctcgtcgtcgctgctcgacgcgatatacaggtccatggaggacgaaccgggccacgccgccggggacgcggcggcgaccaagaagaagcagcagcagcagcagcaggaggaggccctgcactacagctactactacaggccgtcgctggccgggagctaccgtgcgcgcgcgccggccccggggccgcacgccaccacgtccagctcctccgagtgctccagctacggcggcttctcctcgtccgaggccgagtcgtccaaccaccgccgcctgcgccccatccgcaccaccgtgcccgacggccagcccgcgcccgcgccggagaagaaggccaagaagcagccggcgggggcctccatccgcgccaagctcagggacctccgcaagccggcctcccccggcgcccgcctcgcgggcttcctcaacaccatcttcgccggcaagcgcgccccgcagacgccgccctcggcccggacggcggcggagtacgcgtgctcctcggcgtcgtcgtactcgcggtcatgcctgagcaagacgccgtcgacgcgcgggcacgccaaccgcaccgtgcggttcgtggacagcgaccgcgaggcgccggcgacggtgcccggggctgaccgccggagggtgccggtggagcagatgctgctccggcgcatggagatggagagcgacgaggaggacgacgaggagagcagcgacgccagctccgacctgttcgagctggagaacttcgccgcggccgccgtgccgccgggcgcggggtacagggacgagctgccggtgtacgagacgacgagggtggtgctgaaccgcggcattgggcacgcgcagcacgggcacggccggagcgccagagtcgtctga

SEQ ID NO: 51 - protein, 320 aa, Triticum aestivum
MERRGDKGASAAAAARARRHADQPSFSSSLLDAIYRSMEDEPGHPAGDAVTAAATKKKQQQEQEEALHYSYYYRPSLAGSYRARAPAPGPHATTSSSSECSSYGGFSSSEAESSSHRRLRPIRTAVPGGPPAPAPEKKAKKQPAGASIRAKLRDLRKPASPGARLAGFLNTIFAGKRAAPQTPPSARTAAAAEYACSSASSYSRSCLSKTPSTRGHANRTVRFVDSDREAPATVPGADRRRVPVEQMLLRRMEMESDEEDDEDESSDASSDLFELENLAAAAVPPGAGYRDELPVYETTRVVLNPGIGHACGHGRSARVA

SEQ ID NO: 52 - DNA, 963 bp, Triticum aestivum
atggagaggcgcggggacaagggggcgtcggcggcggcggcggcgcgggcgaggcgccacgccgaccagccgtccttctcgtcgtcgctgctggacgcgatatacaggtccatggaggacgagccgggccaccccgctggggacgcggtgactgcggcggctaccaagaagaagcagcagcaggagcaggaggaggccctgcactacagctactactacaggccgtcgctggccgggagctaccgggcgcgcgcgccggcgcccgggccgcacgccaccacgtccagctcctccgagtgctccagctacggcggcttctcctcgtccgaggccgagtcgtccagccaccgccgcctgcgccccatccgcaccgccgtgcccggcgggccgcccgcgcccgcgccggagaagaaggccaagaagcagccggcgggggcctccatccgcgccaagctcagggacctccgcaagccggcctcccccggcgcgcgcctcgcgggcttcctcaacaccatcttcgccggcaagcgcgccgcgccgcagacgccgccctcggcccgcacggcggcggcggcggagtacgcctgctcctcggcgtcgtcctactcgcggtcctgcctgagcaagacgccgtccacgcgcgggcacgccaaccgcaccgtgcggttcgtggacagcgaccgcgaggcgccggcgacggtgcccggggcggaccgccggagggtgccggtggagcagatgctgctccggcgcatggagatggagagcgacgaggaggacgacgaggacgagagcagcgacgccagctcggacctgttcgagctcgagaacttggccgccgccgccgtgccgccgggcgcagggtatcgggacgagctgccggtgtacgagacgaccagggtggtgctgaaccccggcattggccacgcgtgcgggcacggccggagcgccagagtcgcctga

SEQ ID NO: 53 - protein, 430 aa, Glycine max
MDKWDNKPSRKQHHRENPSFSSTLLDVIYRSIDEDPTDEKEEAQLIFYRETMRNQKQSNCFREEKPEAEKHNSRRARKVENWMEKKANEKVVMGRNSLTEFERRTRSNSISNTLSMYSSSTSSESSSVGGFSSSESESFYGVQRPKPIKTSVSDKTKTKTTFDASLHSHNFRSHSSQSQKPKHENGSGKTKSKALKILYGELKKAKQPISPGAKLASFLNSLFTSSGNAKKAKVSTTTTTTTTSTYRPVLIPVATDRTADTKSAAQQQQQPGSTCSSASSFSRSCLSKTPSSRSGAKRSVRFCPVSVIVDEDCRPCGHKNLHEGEESNGKNRSEELRLHVMQESRRVEELARDLLKNYQKKSEVEFDDVMHYEDEEEEEDDDDVASCSSSDLFELDNLSAIGIERYREELPVYETTHFNTNRAIANGFIL

SEQ ID NO: 54 - DNA, 1293 bp, Glycine max
atggacaagtgggacaataaaccctcaagaaaacagcaccacagagaaaacccttctttctcttccactctacttgacgttatctaccgttccatcgacgaagatccaacggatgagaaggaagaagcccaactcatcttctacagagaaaccatgaggaatcagaaacagagcaattgtttcagagaagaaaaacctgaagctgagaaacacaactctcgcagggccaggaaggtggagaattggatggagaagaaagccaacgagaaggttgtaatggggagaaactcattgacggaatttgaacgaagaacgcgaagcaattcaatttcaaacacactctccatgtattcaagctcaacctcttctgagtcaagctctgttggaggcttctcttcttcagagtcagagtccttctatggagtgcaaaggccaaagccaattaaaaccagtgtttctgataaaaccaaaaccaaaacaaccttcgatgcttcacttcacagtcacaacttcaggagccactcttcccaaagccaaaagccaaagcatgagaacggttctggcaaaaccaagtccaaagccttgaaaatcctctatggtgagttgaagaaagcaaagcaaccaatttctccaggtgcaaaacttgctagcttcctcaactctctcttcacttcaagtggaaatgccaagaaagcaaaggtttcaacaacaacaacaacaacaactacgtctacttatcgtcccgtcctgatcccggttgcgacagatcgtactgctgatactaaatcagcagcacaacaacaacaacaacctggttccacttgttcatcagcatcttctttttcaaggtcttgtttgagcaaaaccccttcttcaagatcaggtgcaaaaaggtcagtgaggttttgcccagtgagtgtcatagtggatgaagattgtaggccttgtggtcacaaaaatcttcatgaaggtgaagagagcaatggaaagaacagaagtgaggaacttaggctgcatgtcatgcaggagagtcgcagggtggaggagttagcaagagacttgttgaagaattatcagaaaaagagtgaagtggagtttgatgatgtcatgcattatgaagatgaagaagaagaagaagatgatgatgatgtggctagttgttcaagttctgatctttttgagttggataatctatcagcaattgggattgaaaggtatagggaagagttgcctgtgtatgaaactacccatttcaataccaatagagccattgccaatggcttcattctgtaa

SEQ ID NO: 55 - protein, 431 aa, Glycine max
MDKWDNKPSRKQHHRENPSFSSTLLDVIYRSIDEDPTEEKEEAQLIFYRETMRKQKQGNCFREEKPEAEKHNSRRARKVENWMEKRASEKVLMGRNSLTEFERRTRSNSISNTLSMYSSSTSSESSSVGGFSSSESESFYYGVQRPKPIKTSVSDKTKTKTNFDASLHSHNFRSHSSQSQKPKHENGFGKTKSKALKILYGELKKAKQPISPGAKLASFLNSLFTSTGNAKKAKVSTTTTSTYRPVLIPIATDRVADTKSVSAAQQQPGSTCSSASSFSRSCLSKTPSSRSGAKRSVRFCPVSVIVDEDCRPCGHKNLHEGEESLVDSRGKNRSEELRLHVMQESRRVEELARDLLKNYQKKSEVEFDDVMHYEDEEEEEDDDDVASCASSDLFELDNLSAIGIERYREELPVYETTHFNTNRAIANGFIL

SEQ ID NO: 56 - DNA, 1296 bp, Glycine max
atggacaagtgggacaataaaccctcaagaaaacagcaccacagagaaaacccttcattctcttccactcttcttgacgttatctaccgttccatcgacgaagatccaacggaggagaaggaagaagcccaactcatcttctacagagaaaccatgaggaagcagaaacagggcaattgtttcagagaagaaaaacccgaagctgagaagcacaactctcgcagggccaggaaggtggagaattggatggagaagagagccagcgagaaggttctcatggggagaaactcactgacggaatttgaaagaagaacgcgaagcaattcaatttcaaacacactctccatgtattcaagctcaacctcctctgagtctagctctgttggagggttctcttcttcagagtcagagtccttctattatggagtgcaaaggccaaagccaattaaaaccagtgtttctgataaaaccaaaaccaaaaccaacttcgatgcttcactccacagtcacaacttcaggagccactcttcccaaagccaaaagccaaagcatgagaacggttttggcaaaaccaagtccaaagccttgaaaatcctctatggtgagttgaagaaagcaaagcaaccaatttcacctggtgcaaaacttgctagcttcctcaactctctcttcacttcaactggaaatgccaagaaagcaaaggtttcaacaacaacaacatctacttatcgtcccgtcctgatcccgattgcgacagatcgtgttgctgatactaaatcagtatcagcagcacaacaacaacctggttccacttgttcatcagcatcttctttttcaaggtcttgtttgagcaaaaccccttcttcaagatcaggtgcaaaaaggtctgtgaggttttgtcctgtgagtgtgatagtggatgaagattgcaggccttgtggtcacaagaatcttcatgaaggtgaagagagtttggtggattcccgtggaaagaacagaagtgaggaacttaggctgcatgtcatgcaggagagtcgcagggtggaggagttagcaagagacttgttgaagaattatcagaaaaagagtgaagtggagtttgatgatgtcatgcattatgaagatgaagaagaagaagaagatgatgatgatgtggctagttgtgcaagttctgatctttttgagttggataatctatcagctattgggattgagaggtatagggaagagttgcctgtgtacgaaactacccatttcaataccaatagagccattgccaatggtttcattctgtaa

(19) United States
(12) Patent Application Publication
(10) Pub. No.: US 2023/0357787 A1
(43) Pub. Date: Nov. 9, 2023

(54) COMPOSITIONS AND METHODS FOR IMPROVING GRAIN YIELD IN PLANTS

(71) Applicant: Pioneer Hi-Bred International, Inc., Johnston, IA (US)

(72) Inventors: Bo SHEN, Johnston, IA (US); Wuyi WANG, Newbury Park, CA (US)

(73) Assignee: Pioneer Hi-Bred International, Inc., Johnston, IA (US)

(21) Appl. No.: 17/786,137

(22) PCT Filed: Dec. 17, 2020
(86) PCT No.: PCT/US20/65479

§ 371 Date: Jun. 16, 2022

Related U.S. Application Data
(60) Provisional application No. 62/949,576, filed on Dec. 18, 2019.

(51) Int. Cl. C12N 15/82; C07K 14/415; A01H 6/46

(52) U.S. Cl. CPC C12N 15/8273; C07K 14/415; A01H 6/4684

(57) ABSTRACT

Provided are compositions comprising polynucleotides encoding BG1 polypeptides. Also provided are recombinant DNA constructs, plants, plant cells, seed, grain comprising the polynucleotides, and plants, plant cells, seed, grain comprising a BG1 polypeptide. Additionally, various methods of employing the polynucleotides and genetic modifications in plants, such as methods for increasing BG1 level in a plant and methods for increasing yield of a plant, nutrient utilization efficiency and/or drought tolerance, are also provided herein.

### REFERENCE TO SEQUENCE LISTING SUBMITTED ELECTRONICALLY

The official copy of the sequence listing is submitted electronically via EFS-Web as an ASCII formatted sequence listing with a file named RTS16139E ST25.txt created on Nov. 25, 2019 and having a size of 120 kilobytes and is filed concurrently with the specification. The sequence listing comprised in this ASCII formatted document is part of the specification and is herein incorporated by reference in its entirety.

### FIELD

This disclosure relates to compositions and method for improving yield in plants.

### BACKGROUND

Global demand and consumption of agricultural crops is increasing at a rapid pace. Accordingly, there is a need to develop new compositions and methods to increase yield in plants. This invention provides such compositions and methods.

### SUMMARY

Provided herein are polynucleotides encoding a BG1 polypeptide comprising an amino acid sequence that is at least 90% identical to an amino acid sequence selected from the group consisting of SEQ ID NOs: 1, 3, 5, 7, 9, 11, 13, 15, 17, 19, 21, 23, 25, 27, 29, 31, 33, 35, 41, 43, 45, 47, 49, 51, 53, and 55.

Also provided are recombinant DNA constructs comprising a regulatory element operably linked to a polynucleotide encoding a BG1 polypeptide comprising an amino acid sequence that is at least 90% identical to an amino acid sequence selected from the group consisting of SEQ ID NOs: 1, 3, 5, 7, 9, 11, 13, 15, 17, 19, 21, 23, 25, 27, 29, 31, 33, 35, 41, 43, 45, 47, 49, 51, 53, and 55. In certain embodiments the regulatory element is a heterologous promoter.

Provided are plant cells, plants, and seeds comprising the polynucleotide encoding a BG1 polypeptide or the recombinant DNA construct comprising a regulatory element operably linked to the polynucleotide encoding a BG1 polypeptide. In certain embodiments, the regulatory element is a heterologous promoter. In certain embodiments, the plant and/or seed is from a monocot plant. In certain embodiments, the plant is a monocot plant. In certain embodiments, the monocot plant is maize.

Provided are methods for increasing BG1 activity in a plant by expressing in a regenerable plant cell a recombinant DNA construct comprising a regulatory element operably linked to a polynucleotide encoding a BG1 polypeptide comprising an amino acid sequence that is at least 90% identical to an amino acid sequence selected from the group consisting of SEQ ID NOs: 1, 3, 5, 7, 9, 11, 13, 15, 17, 19, 21, 23, 25, 27, 29, 31, 33, 35, 41, 43, 45, 47, 49, 51, 53, and 55; and generating the plant, wherein the plant comprises in its genome the recombinant DNA construct. In certain embodiments, the regulatory element is a heterologous promoter. In certain embodiments, the plant is a monocot plant. In certain embodiments, the monocot plant is maize.

Provided are methods for improving the drought tolerance or nutrient utilization of a plant by expressing in a regenerable plant cell a recombinant DNA construct comprising a regulatory element operably linked to a polynucleotide encoding a BG1 polypeptide comprising an amino acid sequence that is at least 90% identical to an amino acid sequence selected from the group consisting of SEQ ID NOs: 1, 3, 5, 7, 9, 11, 13, 15, 17, 19, 21, 23, 25, 27, 29, 31, 33, 35, 41, 43, 45, 47, 49, 51, 53, and 55; and generating the plant, wherein the plant comprises in its genome the recombinant DNA construct. In certain embodiments, the regulatory element is a heterologous promoter. In certain embodiments, the plant is a monocot plant. In certain embodiments, the monocot plant is maize.

### BRIEF DESCRIPTION OF THE DRAWINGS

FIG. 1 shows yield advantages of ZM-BG1H1 OE event versus control null. Box plot of hybrid maize yield difference (kg/ha) relative to null non-transgenic hybrid controls for each of the 4 transgenic events across two years of testing. Non-transgenic hybrid control average yield value set to 0 axis. The average yield advantage across all four alleles central dashed line across figure at 355 kg/ha or 5.65 bu/ac. Per each event the average (white line inside each box), 95% confidence interval (black vertical segment adhering right side each box), and outlier values above or below (circles). Significance null hypothesis test (i.e. no difference among the 4 events) not rejected at alpha level 0.05, represented by overlapping rings graph at right.

FIG. 2 shows yield versus control across yield range environments. Hybrid maize yield difference (kg/ha) (Y-axis) relative to null non-transgenic hybrid controls (set to 0 on Y-axis) for each of the 101 tests, comprising the 4 independent ZM-BG1H1 OE events for each test year and location. Non-transgenic hybrid control yield averages (t/ha) at each test location (X-axis). Low-yielding sites below 11.2 t/ha are moderate stress (MS), from 11.2-14.4 t/ha low stress (LS), and above 14.4 t/ha optimal (OPT), these divisions noted by vertical dashed lines and labels at bottom of graph. The average yield advantage at 355 kg/ha is dashed line across figure, as is the 1.0 t/ha reference line. BLUP significance tests are colored: blue, significantly positive (p<0.1); orange, significantly negative (p<0.1); moderate gray, positive non-significant; light gray, negative non-significant. Icon shapes: Event 1, diamond; Event 2, circle; Event 3, star; Event 4, cross.

FIG. 3 is an illustration of secondary agronomic traits correlations to ZM-BG1H1 OE Yield Advantage. Fourteen secondary trait associations to yield advantage in ZM-BG1H1 overexpressing maize plants. See methods for trait definitions. Secondary traits colored by category groupings: canopy or greenness (green); flowering (orange); plant size (dark gray), moisture (blue), yield (maroon). All traits values are averages of all four events, and each converted to percent difference from trait null mean (Y-Axis). All trait percent differences are linearly regressed to the yield percent difference across available field locations and years (up to 101 measurements per trait). The slope of that correlation is projected the X-axis. The R2 of the regression is the icon size. The overall yield difference of 2.4% therefore correlates to itself with a slope of 1.0 and icon size unit size of 1.0 maximum.

FIG. 4 displays results of ear and kernel traits analysis for ZM-BG1H1 OE relative to control. All traits are normalized for comparison to the average percent differences from control average for all the plants across all four events. The standard error bars are derived from the respective individual plant percentage differences from the control average. The t-test significance was done by comparing the set of percentage differences from the control average for the all individual plants across all 4 events, to the set of percent differences among the individual control plants to the control average value.

FIG. 5 shows that ZM-BG1H1 OE increases kernel row number. Histogram distribution of KRN among the four events and control. The percentage of all plants per event or control null are plotted. Note the relative shift of KRN from KRN16 to KRN18 for all four ZM-BG1H1 OE events but a decline for control.

FIG. 6 The average leaf expression of each of the ZM-BG1H1 alleles in V6 greenhouse grown leaves among 416 inbred lines. Haplotype allelic groups were inferred by high resolution genetic marker analysis, and then each haplotype was classified into the five alleles using selected inbred line ZM-BG1H1 gene sequences, including the five inbred lines that generated the reference allelic sequences. The average gene expression levels for each haplotype set are presented. (Haplotypes A1 and A2 are here combined due to ambiguous genetic marker resolution). Standard error bar whiskers for each bar. Horizontal lines across the figure are for the global average (solid line) and StDev (upper-lower dashed lines) for all measurements in the combined set. There is no apparent substantial difference in expression among these allelic haplotypes.

FIG. 7 provides results of hybrid parent seed size (volume, weight, and density). 200 kernel volume (ml), weight (g), and density (g/ml) for control null and average each of the four events. Bars are values mean with standard error whiskers. Horizontal bar across figure is overall mean value and standard deviation for all 4 events and null.

FIG. 8 shows ear and kernel differences at same KRN value. Ear and kernel trait values when the KRN value is normalized. All comparisons to control null are therefore made for the same KRN value, and then the percentage differences across all such comparisons are averaged (gray bars) and juxtaposed next to the equivalent traits percent differences values across all comparison the all aggregate (non-normalized) KRN values (black bars).

FIG. 9 shows the average ear diameter for ZM-BG1H1 OE all events plants (black bars) versus control null (gray bars) across five KRN values. SE bars provided.

### BRIEF DESCRIPTION OF THE SEQUENCE LISTING

The disclosure can be more fully understood from the following detailed description and the accompanying Sequence Listing, which form a part of this application. The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

TABLE 1
Sequence Listing Description (PRT—protein/polypeptide)

| SEQ ID NO | Description | DNA/PRT | Genus species |
|---|---|---|---|
| SEQ ID NO: 1 | ZM-BG1H1A1 BG1 Homolog 1 GRMZM2G178852 | PRT; | Zea mays |
| SEQ ID NO: 2 | ZM-BG1H1A1 BG1 Homolog 1 GRMZM2G178852 | DNA; | Zea mays |
| SEQ ID NO: 3 | ZM-BG1H1A2 BG1 Homolog 1 | PRT; | Zea mays |
| | GRMZM2G178852_allelic_variant | | |
| SEQ ID NO: 4 | ZM-BG1H1A2 BG1 Homolog 1 | DNA; | Zea mays |
| | GRMZM2G178852_allelic_variant | | |
| SEQ ID NO: 5 | ZM-BG1H1A3 BG1 Homolog 1 | PRT; | Zea mays |
| | GRMZM2G178852_allelic_variant | | |
| SEQ ID NO: 6 | ZM-BG1H1A3 BG1 Homolog 1 | DNA; | Zea mays |
| | GRMZM2G178852_allelic_variant | | |
| SEQ ID NO: 7 | ZM-BG1H1A4 BG1 Homolog 1 | PRT; | Zea mays |
| | GRMZM2G178852_allelic_variant | | |
| SEQ ID NO: 8 | ZM-BG1H1A4 BG1 Homolog 1 | DNA; | Zea mays |
| | GRMZM2G178852_allelic_variant | | |
| SEQ ID NO: 9 | ZM-BG1H1A5 BG1 Homolog 1 | PRT; | Zea mays |
| | GRMZM2G178852_allelic_variant | | |
| SEQ ID NO: 10 | ZM-BG1H1A5 BG1 Homolog 1 | DNA; | Zea mays |
| | GRMZM2G178852_allelic_variant | | |
| SEQ ID NO: 11 | ZM-BG1H1A1_MOD1 BG1 Homolog 1 | PRT; | Zea mays |
| | GRMZM2G178852_transgenic_variant | | |
| SEQ ID NO: 12 | ZM-BG1H1A1_MOD1 BG1 Homolog 1 | DNA; | Zea mays |
| | GRMZM2G178852_transgenic_variant | | |
| SEQ ID NO: 13 | ZM-BG1H2A1 ZM-BG1 Family Member | PRT; | Zea mays |
| | GRMZM2G007134 | | |
| SEQ ID NO: 14 | ZM-BG1H2A1 ZM-BG1 Family Member | DNA; | Zea mays |
| | GRMZM2G007134 | | |
| SEQ ID NO: 15 | ZM-BG1H3A1 ZM-BG1 Family Member | PRT; | Zea mays |
| | GRMZM2G438606 | | |
| SEQ ID NO: 16 | ZM-BG1H3A1 ZM-BG1 Family Member | DNA; | Zea mays |
| | GRMZM2G438606 | | |
| SEQ ID NO: 17 | ZM-BG1LH1 ZM-BG1 Family Member | PRT; | Zea mays |
| | GRMZM2G110473 | | |
| SEQ ID NO: 18 | ZM-BG1LH1 ZM-BG1 Family Member | DNA; | Zea mays |
| | GRMZM2G110473 | | |
| SEQ ID NO: 19 | ZM-BG1LH2 ZM-BG1 Family Member | PRT; | Zea mays |
| | GRMZM2G173732 | | |
| SEQ ID NO: 20 | ZM-BG1LH2 ZM-BG1 Family Member | DNA; | Zea mays |
| | GRMZM2G173732 | | |
| SEQ ID NO: 21 | ZM-BG1LH3 ZM-BG1 Family Member | PRT; | Zea mays |
| | GRMZM2G088860 | | |
| SEQ ID NO: 22 | ZM-BG1LH3 ZM-BG1 Family Member | DNA; | Zea mays |
| | GRMZM2G088860 | | |
| SEQ ID NO: 23 | ZM-BG1LH4 ZM-BG1 Family Member | PRT; | Zea mays |
| | GRMZM5G843781 | | |
| SEQ ID NO: 24 | ZM-BG1LH4 ZM-BG1 Family Member | DNA; | Zea mays |
| | GRMZM5G843781 | | |
| SEQ ID NO: 25 | ZM-BG1LH5 ZM-BG1 Family Member | PRT; | Zea mays |
| | GRMZM5G886335 | | |
| SEQ ID NO: 26 | ZM-BG1LH5 ZM-BG1 Family Member | DNA; | Zea mays |
| | GRMZM5G886335 | | |
| SEQ ID NO: 27 | AC-BG1H1 BG1 homolog OAY80775.1 | PRT; | Ananas |
| | | | comosus |
| SEQ ID NO: 28 | AC-BG1H1 BG1 homolog OAY80775.1 | DNA; | Ananas |
| | | | comosus |
| SEQ ID NO: 29 | AT-BG1H1 BG1 homolog AT3G13980.1 | PRT; | Arabidopsis |
| | | | thaliana |
| SEQ ID NO: 30 | AT-BG1H1 BG1 homolog AT3G13980.1 | DNA; | Arabidopsis |
| | | | thaliana |
| SEQ ID NO: 31 | AT-BG1H2 BG1 homolog AT1G54200.1 | PRT; | Arabidopsis |
| | | | thaliana |
| SEQ ID NO: 32 | AT-BG1H2 BG1 homolog AT1G54200.1 | DNA; | Arabidopsis |
| | | | thaliana |
| SEQ ID NO: 33 | BD-BG1H1 BG1 homolog XP_003558688.1 | PRT; | Brachypodium |
| | | | distachyon |
| SEQ ID NO: 34 | BD-BG1H1 BG1 homolog XP_003558688.1 | DNA; | Brachypodium |
| | | | distachyon |
| SEQ ID NO: 35 | HV-BG1H1 BG1 homolog BAJ86540.1 | PRT; | Hordeum |
| | | | vulgare |
| SEQ ID NO: 36 | HV-BG1H1 BG1 homolog BAJ86540.1 | DNA; | Hordeum |
| | | | vulgare |
| SEQ ID NO: 37 | OS-BG1-like BG1 homolog LOC_Os10g25810.1 | PRT; | Oryza sativa |
| SEQ ID NO: 38 | OS-BG1-like BG1 homolog LOC_Os10g25810.1 | DNA; | Oryza sativa |
| SEQ ID NO: 39 | OS-BG1 Rice BIG GRAIN1 OS-BG1 (Q10R09.1) | PRT; | Oryza sativa |
| SEQ ID NO: 40 | OS-BG1 Rice BIG GRAIN1 OS-BG1 (Q10R09.1) | DNA; | Oryza sativa |
| SEQ ID NO: 41 | PD-BG1H1 BG1 homolog XP_008797636.1 | PRT; | Phoenix |
| | | | dactylifera |
| SEQ ID NO: 42 | PD-BG1H1 BG1 homolog XP_008797636.1 | DNA; | Phoenix |
| | | | dactylifera |
| SEQ ID NO: 43 | SB-BG1H1 BG1 homolog XP_021314015.1 | PRT; | Sorghum |
| | | | bicolor |
| SEQ ID NO: 44 | SB-BG1H1 BG1 homolog XP_021314015.1 | DNA; | Sorghum |
| | | | bicolor |
| SEQ ID NO: 45 | SI-BG1H1 BG1 homolog XP_004985512.1 | PRT; | Setaria italica |
| SEQ ID NO: 46 | SI-BG1H1 BG1 homolog XP_004985512.1 | DNA; | Setaria italica |
| SEQ ID NO: 47 | TA-BG1H1 BG1 homolog TRIAE_CS42_4DL_TGACv1 | PRT; | Triticum |
| | | | aestivum |
| SEQ ID NO: 48 | TA-BG1H1 BG1 homolog TRIAE_CS42_4DL_TGACv1 | DNA; | Triticum |
| | | | aestivum |
| SEQ ID NO: 49 | TA-BG1H2 BG1 homolog TRIAE_CS42_4BL_TGACv1 | PRT; | Triticum |
| | | | aestivum |
| SEQ ID NO: 50 | TA-BG1H2 BG1 homolog TRIAE_CS42_4BL_TGACv1 | DNA; | Triticum |
| | | | aestivum |
| SEQ ID NO: 51 | TA-BG1H3 BG1 homolog TRIAE_CS42_4AS_TGACv1 | PRT; | Triticum |
| | | | aestivum |
| SEQ ID NO: 52 | TA-BG1H3 BG1 homolog TRIAE_CS42_4AS_TGACv1 | DNA; | Triticum |
| | | | aestivum |
| SEQ ID NO: 53 | GM-BG1H1 BG1 homolog Glyma.07G036700.1 | PRT; | Glycine max |
| SEQ ID NO: 54 | GM-BG1H1 BG1 homolog Glyma.07G036700.1 | DNA; | Glycine max |
| SEQ ID NO: 55 | GM-BG1H2 BG1 homolog Glyma.16G006100.1 | PRT; | Glycine max |
| SEQ ID NO: 56 | GM-BG1H2 BG1 homolog Glyma.16G006100.1 | DNA; | Glycine max |

### DETAILED DESCRIPTION

### I. Compositions

### A. BG1 Polynucleotides and Polypeptides

The present disclosure provides polynucleotides encoding BG1 polypeptides. Maize BG1 polypeptides comprise a unique plant specific gene family. The BG1 protein family analysis of describes a gene family of proteins with an N-terminal region rich in glutamic acid and aspartic acid repeats but without an ordered structural propensity, and a conserved C-terminal region without significant similarity to other characterized functional domains. As used herein Maize BG1 “polypeptide,” “protein,” or the like, refers to a protein with domain structures similar to other BG1 related proteins, represented by a general structure of SEQ ID NOS: 1, 3, 5, 7, 9, 11, 13, 15, 17, 19, 21, 23, 25 or a sequence that is at least 90-100% identical to one of the aforementioned sequences.

One aspect of the disclosure provides a polynucleotide encoding a BG1 polypeptide comprising an amino acid sequence that is at least 90% identical to the amino acid sequence of any one of SEQ ID NOs: 1, 3, 5, 7, 9, 11, 13, 15, 17, 19, 21, 23, 25, 27, 29, 31, 33, 35, 41, 43, 45, 47, 49, 51, 53, and 55. In certain embodiments, the polynucleotide encoding a BG1 polypeptide comprises an amino acid sequence that is at least 95% identical to the amino acid sequence of any one of SEQ ID NOs: 1, 3, 5, 7, 9, 11, 13, 15, 17, 19, 21, 23, 25, 27, 29, 31, 33, 35, 41, 43, 45, 47, 49, 51, 53, and 55.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98).

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, California, USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, California, GAP, BESTFIT, BLAST, FASTA and TFASTA in the Wisconsin Genetics Software Package®, Version 8 (available from Genetics Computer Group (GCG® programs (Accelrys, Inc., San Diego, CA)). The CLUSTAL program is well described by Higgins and Sharp, (1988) Gene 73:237 44; Higgins and Sharp, (1989) CABIOS 5:151 3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

Accordingly, in any of the embodiments described herein, the BG1 polynucleotide may encode a BG1 polypeptide that is at least 80% identical to any one of SEQ ID NOs: 1, 3, 5, 7, 9, 11, 13, 15, 17, 19, 21, 23, 25, 27, 29, 31, 33, 35, 41, 43, 45, 47, 49, 51, 53, and 55. For example, the BG1 polynucleotide may encode a BG1 polypeptide that is at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, or at least 99% identical to the amino acid sequence of any one of SEQ ID NOs: 1, 3, 5, 7, 9, 11, 13, 15, 17, 19, 21, 23, 25, 27, 29, 31, 33, 35, 41, 43, 45, 47, 49, 51, 53, and 55.

### B. Recombinant DNA Construct

Also provided is a recombinant DNA construct comprising any of the BG1 polynucleotides described herein. In certain embodiments, the recombinant DNA construct further comprises at least one regulatory element. In certain embodiments, the at least one regulatory element of the recombinant DNA construct comprises a promoter. In certain embodiments, the promoter is a heterologous promoter.

As used herein, a “recombinant DNA construct” comprises two or more operably linked DNA segments, preferably DNA segments that are not operably linked in nature (i.e., heterologous). Non-limiting examples of recombinant DNA constructs include a polynucleotide of interest operably linked to heterologous sequences, also referred to as “regulatory elements,” which aid in the expression, autologous replication, and/or genomic insertion of the sequence of interest. Such regulatory elements include, for example, promoters, termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The BG1 polynucleotides described herein can be provided for expression in a plant of interest or any organism of interest. The cassette can include 5′ and 3′ regulatory sequences operably linked to a BG1 polynucleotide. “Operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the BG1 polynucleotide to be under the transcriptional regulation of the regulatory regions. The expression cassette may additionally contain selectable marker genes.

The expression cassette can include in the 5′-3′ direction of transcription, a transcriptional and translational initiation region (e.g., a promoter), a BG1 polynucleotide, and a transcriptional and translational termination region (e.g., termination region) functional in plants. The regulatory regions (e.g., promoters, transcriptional regulatory regions, and translational termination regions) and/or the BG1 polynucleotide may be native/analogous to the host cell or to each other. Alternatively, the regulatory regions and/or the BG1 polynucleotide may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, with the plant host, or may be derived from another source (i.e., foreign or heterologous) than the promoter, the BG1 polynucleotide, the plant host, or any combination thereof.

The expression cassette may additionally contain a 5′ leader sequences. Such leader sequences can act to enhance translation. Translation leaders are known in the art and include viral translational leader sequences.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein “promoter” refers to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Certain types of promoters preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Also contemplated are synthetic promoters which include a combination of one or more heterologous regulatory elements.

The promoter of the recombinant DNA constructs of the invention can be any type or class of promoter known in the art, such that any one of a number of promoters can be used to express the various BG1 polynucleotide sequences disclosed herein, including the native promoter of the polynucleotide sequence of interest. The promoters for use in the recombinant DNA constructs of the invention can be selected based on the desired outcome.

### C. Plants and Plant Cells

Provided are plants, plant cells, plant parts, seed, and grain comprising a BG1 polynucleotide sequence described herein or a recombinant DNA construct described herein, so that the plants, plant cells, plant parts, seed, and/or grain have increased expression of a BG1 polypeptide. In certain embodiments, the plants, plant cells, plant parts, seeds, and/or grain have stably incorporated a BG1 polynucleotide described herein into its genome. In certain embodiments, the plants, plant cells, plant parts, seeds, and/or grain can comprise multiple BG1 polynucleotides (i.e., at least 1, 2, 3, 4, 5, 6 or more).

In specific embodiments, the BG1 polynucleotides in the plants, plant cells, plant parts, seeds, and/or grain are operably linked to a heterologous regulatory element, such as but not limited to a constitutive promoter, a tissue-preferred promoter, or a synthetic promoter for expression in plants or a constitutive enhancer.

A “genomic locus” as used herein, generally refers to the location on a chromosome of the plant where a gene, such as a polynucleotide encoding a BG1 polypeptide, is found. As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

A “regulatory element” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located.

An “enhancer” element is any nucleic acid molecule that increases transcription of a nucleic acid molecule when functionally linked to a promoter regardless of its relative position.

A “repressor” (also sometimes called herein silencer) is defined as any nucleic acid molecule which inhibits the transcription when functionally linked to a promoter regardless of relative position.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene but is not necessarily a part of the sequence that encodes the final gene product.

The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Genetic modification,” “DNA modification,” and the like refers to a site-specific modification that alters or changes the nucleotide sequence at a specific genomic locus of the plant. The genetic modification of the compositions and methods described herein may be any modification known in the art such as, for example, insertion, deletion, single nucleotide polymorphism (SNP), and or a polynucleotide modification. Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region.

As used herein, a “targeted” genetic modification or “targeted” DNA modification, refers to the direct manipulation of an organism's genes. The targeted modification may be introduced using any technique known in the art, such as, for example, plant breeding, genome editing, or single locus conversion.

The type and location of the DNA modification of the BG1 polynucleotide is not particularly limited so long as the DNA modification results in increased expression and/or activity of the protein encoded by the BG1 polynucleotide.

In certain embodiments, the plant, plant cells, plant parts, seeds, and/or grain comprise one or more nucleotide modifications present within (a) the coding region; (b) non-coding region; (c) regulatory sequence; (d) untranslated region, or (e) any combination of (a)-(d) of an endogenous polynucleotide encoding a BG1 polypeptide.

As used herein “increased,” “increase,” or the like refers to any detectable increase in an experimental group (e.g., plant with a DNA modification described herein) as compared to a control group (e.g., wild-type plant that does not comprise the DNA modification. Accordingly, increased expression of a protein comprises any detectable increase in the total level of the protein in a sample and can be determined using routine methods in the art such as, for example, Western blotting and ELISA.

In certain embodiments, the genomic locus has more than one (e.g., 2, 3, 4, 5, 6, 7, 8, 9, or 10) DNA modification. For example, the translated region and a regulatory element of a genomic locus may each comprise a targeted DNA modification. In certain embodiments, more than one genomic locus of the plant may comprise a DNA modification.

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides or genetic modification(s).

The polynucleotides or recombinant DNA constructs disclosed herein may be used for transformation of any plant species, including, but not limited to, monocots and dicots. Additionally, the genetic modifications described herein may be used to modify any plant species, including, but not limited to, monocots and dicots.

Examples of plant species of interest include, but are not limited to, maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum.

Vegetables include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Other plants of interest include, for example, grain plants that provide seeds of interest, oil-seed plants, and leguminous plants. Seeds of interest include, for example, grain seeds, such as corn, wheat, barley, rice, sorghum, rye, etc. Oil-seed plants include, for example, cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea.

For example, in certain embodiments, maize plants are provided that comprise, in their genome, a polynucleotide that encodes a BG1 polypeptide comprising an amino acid sequence that is at least 90% identical to any one of SEQ ID NOs: 1, 3, 5, 7, 9, 11, 13 and 15.

D. Stacking Other Traits of Interest

In some embodiments, the inventive BG1 polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glufosinate herbicides; herbicides which target the protox enzyme (also referred to as “protox inhibitors”).

The plant, plant cell, plant part, seed, and/or grain having an inventive polynucleotide sequence can also be combined with at least one other trait to produce plants that further comprise a variety of desired trait combinations. For instance, the plant, plant cell, plant part, seed, and/or grain having an inventive polynucleotide sequence may be stacked with polynucleotides encoding polypeptides having pesticidal and/or insecticidal activity, or a plant, plant cell, plant part, seed, and/or grain having an inventive polynucleotide sequence may be combined with a plant disease resistance gene.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Any plant having an inventive polynucleotide sequence disclosed herein can be used to make a food or a feed product. Such methods comprise obtaining a plant, explant, seed, plant cell, or cell comprising the polynucleotide sequence and processing the plant, explant, seed, plant cell, or cell to produce a food or feed product.

### II. Methods of Use

A. Methods for Increasing Yield, Increasing Drought Tolerance, and/or Increasing the Activity of BG1 in a Plant

Provided are methods for increasing yield in a plant, increasing drought tolerance of a plant, increasing lateral root development, and/or increasing the activity of BG1 in a plant comprising introducing into a plant, plant cell, plant part, seed, and/or grain a recombinant DNA construct comprising any of the inventive polynucleotides described herein, whereby the polypeptide is expressed in the plant. Also provided are methods for increasing yield in a plant, increasing drought tolerance of a plant, and/or increasing the activity of BG1 in a plant comprising introducing a genetic modification at a genomic locus of a plant that encodes a BG1 polypeptide comprising an amino acid sequence that is at least 90% identical to the amino acid sequence set for in any one of SEQ ID NOs: 1, 3, 5, 7, 9, 11, 13, 15, 17, 19, 21, 23, 25, 27, 29, 31, 33, 35, 41, 43, 45, 47, 49, 51, 53, and 55.

The plant for use in the inventive methods can be any plant species described herein. In certain embodiments, the plant is a grain plant, an oil-seed plant, or leguminous plant. In certain embodiments, the plant is a grain plant such as maize.

As used herein, “yield” refers to the amount of agricultural production harvested per unit of land and may include reference to bushels per acre of a crop at harvest, as adjusted for grain moisture (e.g., typically 15% for maize). Grain moisture is measured in the grain at harvest. The adjusted test weight of grain is determined to be the weight in pounds per bushel, adjusted for grain moisture level at harvest.

As used herein, “Drought tolerance” refers to a trait of a plant to survive under drought conditions over prolonged periods of time without exhibiting substantial physiological or physical deterioration.

“Increased drought tolerance” of a plant refers to any measurable improvement in a physiological or physical characteristic, such as yield, as measured relative to a reference or control plant. Typically, when a plant comprising a recombinant DNA construct or DNA modification in its genome exhibits increased drought tolerance relative to a reference or control plant, the reference or control plant does not comprise in its genome the recombinant DNA construct or DNA modification.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

As used herein, increase in BG1 activity, refers to any detectable increase in the activity of the BG1 protein compared to a suitable control. The BG1 activity may be any known biological property and includes, for example, increased formation of protein complexes and/or modulation of biochemical pathways.

Various methods can be used to introduce a sequence of interest into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

“Stable transformation” is intended to mean that the polynucleotide introduced into a plant integrates into the genome of the plant of interest and is capable of being inherited by the progeny thereof. “Transient transformation” is intended to mean that a polynucleotide is introduced into the plant of interest and does not integrate into the genome of the plant or organism or a polypeptide is introduced into a plant or organism.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osj oda et al. (1996) Nature Biotechnology 14:745-750 (maize via Agrobacterium tumefaciens); all of which are herein incorporated by reference.

In specific embodiments, the BG1 sequences can be provided to a plant using a variety of transient transformation methods. Such transient transformation methods include, but are not limited to, the introduction of the BG1 protein directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

One of skill will recognize that after the expression cassette containing the inventive polynucleotide is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included, provided that these parts comprise cells comprising the inventive polynucleotide. Progeny and variants, and mutants of the regenerated plants are also included, provided that these parts comprise the introduced nucleic acid sequences.

In one embodiment, a homozygous transgenic plant can be obtained by sexually mating (selfing) a heterozygous transgenic plant that contains a single added heterologous nucleic acid, germinating some of the seed produced and analyzing the resulting plants produced for altered cell division relative to a control plant (i.e., native, non-transgenic). Back-crossing to a parental plant and out-crossing with a non-transgenic plant are also contemplated.

Therefore, in certain embodiments the method comprises: (a) expressing in a regenerable plant cell any of the inventive polynucleotides described herein, e.g., a recombinant DNA construct comprising a polynucleotide encoding an amino acid sequence that is at least 90% identical to the amino acid sequence of any one of SEQ ID NOs: 1, 3, 5, 7, 9, 11, 13, 15, 17, 19, 21, 23, 25, 27, 29, 31, 33, 35, 41, 43, 45, 47, 49, 51, 53, and 55, and (b) generating the plant, wherein the plant comprises in its genome the recombinant DNA construct of interest.

Methods for transforming dicots, primarily by use of Agrobacterium tumefaciens, and obtaining transgenic plants have been published, among others, for cotton (U.S. Pat. Nos. 5,004,863, 5,159,135); soybean (U.S. Pat. Nos. 5,569,834, 5,416,011); Brassica (U.S. Pat. No. 5,463,174); peanut (Cheng et al., Plant Cell Rep. 15:653 657 (1996), McKently et al., Plant Cell Rep. 14:699 703 (1995)); papaya (Ling et al., Bio/technology 9:752 758 (1991)); and pea (Grant et al., Plant Cell Rep. 15:254 258 (1995)). For a review of other commonly used methods of plant transformation see Newell, C. A., Mol. Biotechnol. 16:53 65 (2000). One of these methods of transformation uses Agrobacterium rhizogenes (Tepfler, M. and Casse-Delbart, F., Microbiol. Sci. 4:24 28 (1987)). Transformation of soybeans using direct delivery of DNA has been published using PEG fusion (PCT Publication No. WO 92/17598), electroporation (Chowrira et al., Mol. Biotechnol. 3:17 23 (1995); Christou et al., Proc. Natl. Acad. Sci. U.S.A. 84:3962 3966 (1987)), microinjection, or particle bombardment (McCabe et al., Biotechnology 6:923-926 (1988); Christou et al., Plant Physiol. 87:671 674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press, Inc.: San Diego, CA, 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

The following are examples of specific embodiments of some aspects of the invention. The examples are offered for illustrative purposes only and are not intended to limit the scope of the invention in any way.

### EXAMPLE 1

### BG1 Gene Family Identification and Characterization

Maize genomes and transcriptomes were searched and identified 10 candidate maize family members. There are 8 members of the BG1-related gene family in maize with over 20% amino acid identity (AAID) to OS-BG1 (Table 2). One gene GRMZM2G027519 on genome draft RefGen2, is identical to GRMZM5G843781 on chr. 7, and only the chr. 7 locus remains in the newer AGPv4 genome draft.

TABLE 2
BG1 and BG1-Like Family Members

| | | | AAID | AASIM | |
|---|---|---|---|---|---|
| Gene | Locus | AA | (%) | (%) | Chr. |
| ZM-BG1H1 | GRMZM2G178852 | 316 | 65.1 | 71.6 | 1 |
| ZM-BG1H2 | GRMZM2G007134 | 311 | 57.6 | 65.1 | 9 |
| ZM-BG1H3 | GRMZM2G438606 | 312 | 56.3 | 64.6 | 9 |
| ZM-BG1LH1 | GRMZM2G110473 | 333 | 40.8 | 49.9 | 5 |
| ZM-BG1LH2 | GRMZM2G173732 | 311 | 39.3 | 48.9 | 1 |
| ZM-BG1LH3 | GRMZM2G088860 | 350 | 25.6 | 36.6 | 2 |
| ZM-BG1LH4 | GRMZM5G843781 | 347 | 23.9 | 36.9 | 7 |
| ZM-BG1LH5 | GRMZM5G886335 | 324 | 22.4 | 30.3 | 5 |

Gene name, public locus name, peptide length (amino acids), chromosome locations, and global amino acid identity (AAID) and similarity (AASIM) to rice OS-BG1. The closest homolog by protein relationship to OS-BG1 (65.1% identity) is locus GRMZM2G178852, which we name Zea mays BIG GRAIN1 Homolog 1 (ZM-BG1H1). The second closest homolog to OS-BG1 (56.3%-57.6% identity) is a single or duplicated gene locus on chromosome 9. In the B73 genomic assemblies RefGen2.0 or AGPv4.0 this region is represented by two very closely-related (97.8% AAID) and closely-spaced loci GRMZM2G007134 (ZM-BG1H2) and GRMZM2G438606 (ZM-BG1H3). In the public genome drafts RefGen2 and AGPv4 the region between these two genes is gap-filled with a 50 kb N-spacer. A proprietary genome draft of a different stiff-stalk line indicates that these two genes are linked 31.5 kb apart ATG-ATG in arrangement indicating direct regional tandem duplication, with the variant GRMZM2G438606 the most distal (telomeric) of the two genes. In some proprietary non-stiff-stalk lines genome drafts, however, this region appears as a single copy of locus GRMZM2G438606, suggesting this locus may have been duplicated (or preferentially retained) to present GRMZM2G007134 in only a subset of maize lineages. Gene expression and genetic haplotype analyses (below) for this complex locus pair likely conflate these two loci because they are 99.3% nt identical in the ORF and very closely spaced, and thus we will commonly refer to them together as ZM-BG1H2(3). The ZM-BG1H1 gene has about 65% AAID to the ZM-BG1H2(3) gene pair.

Two other more distantly related genes, ZM-BG1LH1 (GRMZM2G110473) and ZM-BG1LH2 (GRMZM2G110473) (for Zea mays BG1-like homologs 1 and 2), have 41.1 and 39.3% AAID to OS-BG1, but somewhat higher amino acid similarity, 54.4 and 49.6% respectively, to the OS-BG1-like gene locus (LOC_Os10g25810.1). The BG1 family is divided into major clades separating BG1 homologs and BG1-like homologs. These two genes are classified as BG1-like. The two maize genes are 73.8% AAID, indicating they recently duplicated. Three other BG1-Like genes ZM-BG1LH3, ZM-BG1LH4 and ZM-BG1LH5 have very low (less than 26%) amino acid similarity to OS-BG1. ZM-BG1LH3 and ZM-BG1LH4 are a pair sharing 74.9% AAID, whereas ZM-BG1H5 is the most distinct sharing less than 23% ID to all other family members (Table 2).

ZM-BG1H1 and the ZM-BG1H2(3) pair was identified as candidate OS-BG1 orthologs. Chromosomes 1 and 9 share large regions of intra-genomic synteny. The local Chr. 1 region surrounding ZM-BG1H1 shares multiple gene homologs to the genes in the region Chr. 9 around ZM-BG1H2(3). And just as ZM-BG1H1 and ZM-BG1H2(3) are in opposite directions on their respective chromosomes, reverse and forward respectively, the relative gene order in their local syntenic homologous gene neighbors is also inverted. Sorghum has only one OS-BG1 homolog, and while it has higher identity to ZM-BG1H1 (77.5%) than to ZM-BG1H2(3) (69.6%), the sequence is intermediate between the two. This indicates the maize-sorghum last common ancestor (ca. 11.9 m.y.a.) likely had a single BG1 homolog gene, and that the genome duplication event at ca. >4.8 m.y.a., resulted in the maize loci on chromosomes 1 and 9, but other gene loss/retention scenarios since the maize-sorghum pre-ancestor are possible.

### EXAMPLE 2

### Gene Expression Analyses

The ZM-BG1 family was analyzed for gene expression using a set of 755 B73 RNAseq samples generated. The OS-BG1 showed highest levels of expression in the shoot apical meristem and in the developing inflorescence, but lower levels in developing seeds, lower still in leaves and roots (see Rice eFP Browser at bar.utoronto.ca, querying alias LOC_0s03g07920). The maize gene family expression patterns were observed across 755 diverse tissue-treatment mRNA profiling samples organized into five major tissue categories. ZM-BG1 gene family mRNA expression was performed across five major maize tissues categories (Root, Green Tissue, Meristem, Ear, and Tassel) from a B73-based Gene Expression Atlas. Expression values were measured in mean pptm (parts per 10 million) for each tissue category. The highest average expression across all samples is ZM-BG1H1. Zm-BG1H2(3) expression patterns are not distinguished because they are 99.3% nt identical, but it appears they collectively have lower levels of expression than ZM-BG1H1, although in some tissues the public eFP browser indicates ZM-BG1H2(3) has higher expression. The remaining family members have even lower expression levels.

TABLE 3
Endogenous and Transgene ZM-BG1H1
Gene Expression Levels Compared

| | Location | | | |
|---|---|---|---|---|
| | Growth Chamber | | Field | |
| | Gene | | | |
| | ZM-BG1H1 | ZM-BG1H1 | ZM-BG1H1 | ZM-BG1H1 |
| | (Native) | (MOD1) | (Native) | (MOD1) |
| | Native | Relative | Native | Relative |
| Event | Expression | Increase | Expression | Increase |
| Event_1 | 5.7 | 47 | 3.2 | 28 |
| Event_2 | 4.6 | 96 | 3.2 | 33 |
| Event_3 | 5.7 | 42 | 3.3 | 35 |
| Event_4 | 4.9 | 41 | 4.1 | 33 |
| Ave 4 Events | 5.2 | 57 | 3.4 | 32 |
| Null | 4.2 | na | 2.2 | na |
| Event/Null | 1.2 | | 1.6 | |

Endogenous native ZM-BG1H1 mRNA expression was measured across all four events and control null, indicating the native gene expression varied between events and null. Separately, an estimation of the transgene ZM-BG1H1 (MOD1) expression relative to the native endogenous ZM-BG1H1 expression. The PCR primers and assay are distinct for the native versus transgene ZM-BG1H1 (MOD1) which distinguish their expression. The relative fold-increased expression of the ZM-BG1H1 (MOD1) transgene relative to endogenous ZM-BG1H1 native gene was estimated by comparison to a common internal constitutive control in each assay.

Focusing in on ZM-BG1H1 vs ZM-BG1H2(3), through a finer resolution of tissue patterns, Zm-BG1H1 is observed to have highest expression in stalks, immature ear, silk and tassel, whereas ZM-BG1H2(3) has its highest expression in husk and immature ear.

ZM-BG1H1 gene versus ZM-BG1H2(3) gene(s) expression comparison in greater detail. Gene expression in 19 tissue categories were from the B73-based Gene Expression Atlas. Leaf diurnal gene expression between ZM-BG1H1 gene versus ZM-BG1H2(3) gene(s) was performed. ZM-BG1H1 possesses marked diurnal (day-night) expression with peak at ZT14 or early evening. ZM-BG1H1 expression exceeds ZM-BG1H2(3) combined levels day or night. ZM-BG1H1, ZM-BG1H2(3). In all tissues but husk, ear leaf sheath and pericarp, Zm-BG1H1 has higher expression. The maize eFP browser comparison shows ZM-BG1H1 has its highest expression in stems and shoot apical meristem, cob, tassel, and silks, and for ZM-BG1H2(3) the highest expression is in cob, endosperm, kernels, and husk. In the eFP leaf gradient expression patterns, both genes show leaf expression concentrated in the basal half of the leaf, with some extreme tip expression, especially for ZM-BG1H2(3). These tissue expression patterns do not completely resolve which gene is most similar in native expression pattern to OS-BG1, but ZM-BG1H1 has especially higher expression in meristematic tissues and developing inflorescences, which matches the expression pattern for OS-BG1. ZM-BG1H1 and other members of the BG1 family do not show high leaf or green tissue expression. This could be due in part to most samples being harvested during the day. A plot of the diurnal expression patterns for ZM-BG1H1 and ZM-BG1H2(3) was made. ZM-BG1H1 reveals a distinct diurnal pattern with highest expression in the evening dark.

The set of 755 RNA-seq transcript samples was used to determine genes correlated to ZM-BG1H1 and ZM-BG1H2(3) gene expression using Pearson's correlation (r-value) at 0.7 and a minimum expression level of at least 5 pptm in two or more samples. For ZM-BG1H1 a set of 136 transcripts were correlated, and among these correlated transcripts, the top enriched 15 Gene Ontological terms includes nucleosome, nucleolus, nucleus and DNA binding, as well as thylakoid and chloroplast, plasmodesma, vacuolar and plasma membrane, and cell division and cell cycle. In comparison, ZM-BG1H2(3) had 101 correlated transcripts, with nucleus and transcription topping the list, but these GO term enrichment values are much less significant than for GO terms enriched for ZMBG1H1.

### EXAMPLE 3

### Transgene Event Evaluation and Field Yield Tests

The maize gene ZM-BG1H1 gene was chosen for transgenic OE with ZM-GOS2 PRO in maize, using the B73 reference allele ZM-BG1H1-A1, the most common among SS lines, albeit with two amino acid and ORF nt changes described, hence ZM-BG1H1 (MOD1). The ZM-GOS2 PRO confers moderate constitutive expression. The elite germplasm non-stiff-stalk inbred line PH184C was used for transformation, which possesses the ZM-BG1H1A3 allele common among NSS lines. Southern-by-Sequencing was used to evaluate the uniqueness locations of the four events. Events 1, 3 and 4 mapped to chromosome 2 but at separate locations, respectively, in the B73 genome draft RefGen2 at positions Chr2:120.4Mbp, Chr2:1.3Mbp, and Chr2:164.7Mbp. Event 2 was assigned to a distinct region not present in the B73 genome but matching the transformed line PH184C genome. The T1 generation plants were top-crossed to line PHW3G which is a stiff-stalk variety possessing the ZM-BG1H1-A1/2 allele.

Transgene expression was assayed by qRT-PCR first at the T0 generation for the event selections, and again later, as hybrid seed used for the yield test. The relative expression of the endogenous ZM-BG1H1 gene versus the ZM-BG1H1 (MOD1) transgene was compared in growth chamber hybrid V3-V4 seedling leaves, and again in field grown R1 mature ear leaves. Both indicate that the transgene events have marked detectable ZM-BG1H1 (MOD1) expression, estimated to be about 1000-2000 pptm by comparing to the qRT-PCR internal constitutive control GRMZM5G877316_T02 to its benchmark expression in the gene expression atlas. In the growth-chamber plants the ZM-BG1H1 (MOD1) is elevated in expression, relative to the ZM-BG1H1 native locus, by an estimated average >57-fold across all four events, and in the field-grown plants by >32-fold (Table 2). This is an inferred relative fold change because the ZM-BG1H1 native gene and ZM-BG1H1 MOD1 transgene involve distinct qRT-PCR assays. Their relative expression was estimated by comparison of each to a common internal gene PCR control, the broadly expressed gene transcript GRMZM5G877316_T02. Because the native gene is expressed at very low levels, even modest background qRT-PCR signal in the native gene assay could lead to underestimation of the relative fold-change induction for the transgene. Although the transgene uses specific isolated DNA fragment of the ZM-GOS2 PRO, when comparing the relative endogenous native gene expression levels between ZM-GOS2 (GRMZM2G073535) and ZM-BG1H1 using 468 RNASeq B73 samples, ZM-GOS2 gene expression averaged 375-fold higher expression than ZM-BG1H1. When broken down by 11 major tissue types, the ratio ranged from 553- and 541-fold higher in Leaf/Shoot and Endosperm respectively, to 21-fold and 18-fold in Tassel and Stem/Stalk respectively. The average leaf tissue expression of ZM-GOS2 was 6500 pptm, 3 to 6-fold higher than the RT-PCR transgene estimate above. These results also demonstrate that the native ZM-GOS2 expression is not only higher in expression than ZM-BG1H1, but that it also has a distinctive tissue-spatial-temporal pattern relative to native ZM-BG1H1.

The ZM-BG1H1 OE events (E1-E4) were field tested for yield in comparison to the non-transgenic null control in multiple field locations and environments over two years of tests. These yield tests were conducted across a total of 26 site locations, which across both years produced a range of yield environments, with the control yields ranging from 9.4 to 17.4 t/ha.

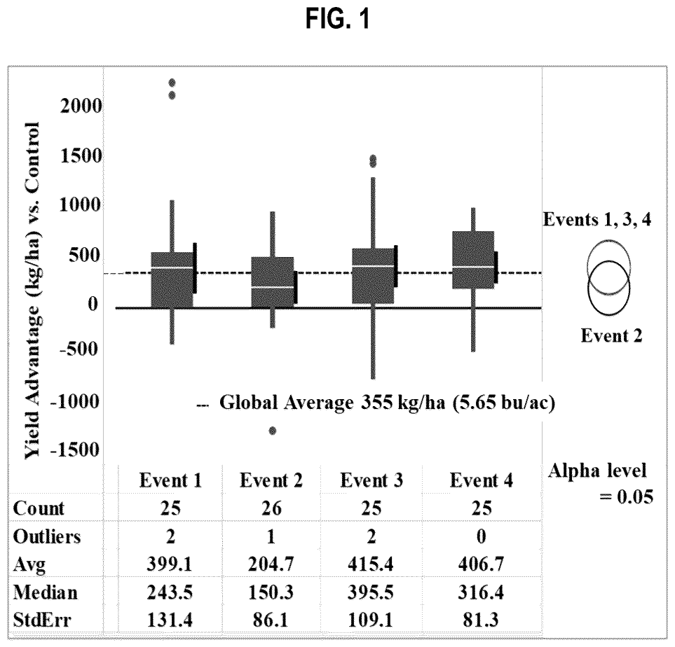

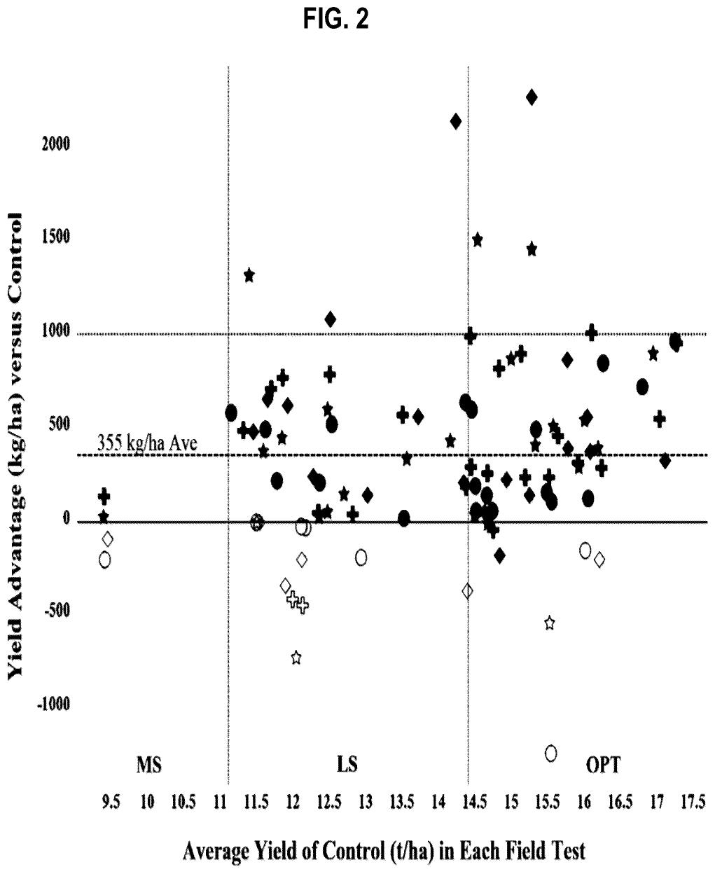

These sites were chosen to provide environment and stress variations generally, with water availability stress is a common driver of yield difference across these sites. The lowest yielding environments below 11.2 t/ha were classified as moderate stress, those from 11.2-14.4 t/ha light stress, and all those above 14.4 t/ha were classified as optimal growth conditions. All four events increased yield per unit area relative to controls across both years, with an overall test average of 355 kg/ha (5.65 bu/ac) (FIG. 1). The event performance ranged from 204.7 kg/ha for event E2, and 399.1, 406.7 and 415.4 kg/ha for events E1, E4, and E3 respectively. Event-to-event variation was small, no difference at not rejected at alpha 0.05 significance test. Event 2 lags, but events E1, E3, and E4 are indistinguishable at alpha 0.05, average 407 kg/ha (6.5 bu/ac) advantage. The yield differences for all 101 event-location-year tests are shown in FIG. 2. Eighty-three percent of the tests were nominally positive, with 29 statistically significant at BLUP P-value 0.1, with only two of the negative yielding values statistically significant at BLUP P-value 0.1. Seven of the tests yielded over 1 ton per hectare advantage. The four events were spread across the performance spectrum, with all four events having representatives in either the upper or lower 10% yield difference values. The ZM-BG1H1 OE tests showed yield advantage across the wide range of environments encompassing light stress to optimal conditions. There was little or no advantage under moderate stress, but this is based upon only one location. Linear regression analysis of yield advantage relative to control yield was only r2=0.05, indicating little co-association. This indicates that ZM-BG1H1 OE conferred yield advantages across a broad range of environments, test locations, and stress levels FIG. 2.

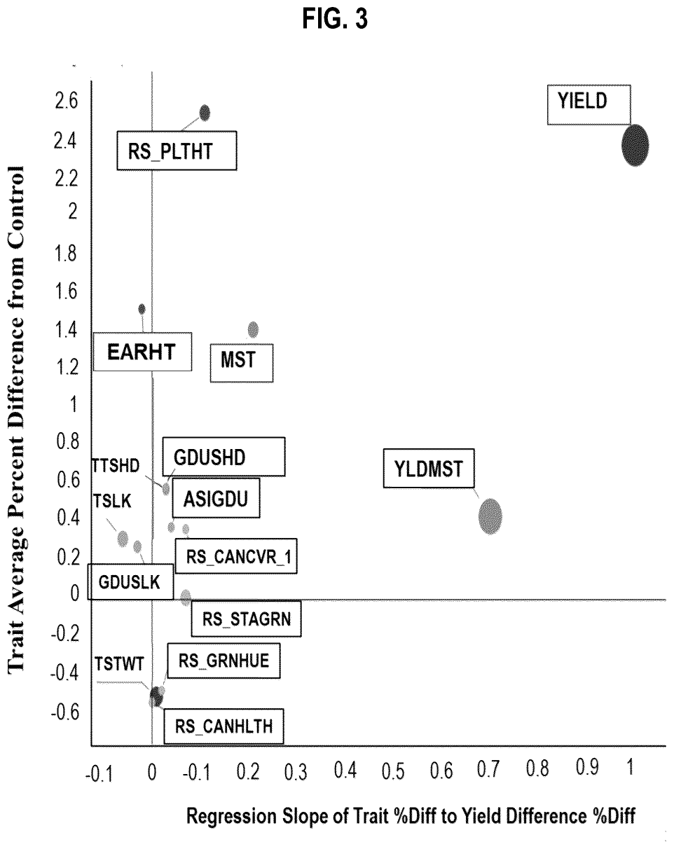

The ZM-BG1H1 OE events were assessed by a combination of aerial and ground observations in the field tests for differences from the control for a set of agronomic traits relevant to maize breeding. These included traits spanning flowering, canopy and vegetative greenness, plant size and architecture, and grain moisture. All these traits including yield were converted to percent differences from the control to enable trait-to-trait comparisons. A linear regression analysis of each trait to yield advantage ZM-BG1H1 at each was calculated (all events combined). The percent differences from control, and the yield difference correlation slope and regression correlation for each of these traits are plotted together in FIG. 3. Yield advantage as the reference trait has a slope of 1 and correlates to itself. The four canopy greenness traits overall showed little differences from the control, and little organized slope or correlation in relation to yield. The four flowering time measures trended slightly positive versus the control (ranging 0.3% to 0.6% differences) but they showed effectively no positive slope or correlation with the yield advantage. Both plant height and ear height were above control, 2.6% and 1.5% respectively, however both also showed little to no positive slope or correlation to yield advantage. Grain moisture (MST) was slightly higher than the control (1.4%) and showed a slight positive slope and co-association with yield (r2=0.19). When moisture was combined with yield (YLDMST, or yield per moisture), as expected the correlation to yield was more positive and significant (slope 0.7 and r2=0.8). Grain density (TSTWT) was down an average 0.5% (slope 0.01, r2=0.31).

Flowering Time: The four events and control were replanted in a dedicated observation plot in year 3 (Yr3-Obs) to both confirm or extend the phenotypic observations made in the yield trials. No differences from control in germination, seedling stand count, canopy closure, leaf size shape or color, tillers, and plant height were observed through V11 when the plants were at 1.8 m height. Flowering measurements began at 62 days (1353 GDU growth heat units) after planting and proceeded daily through day 68 (1488 GDU). A flowering graph plot for control and each event was used to interpolate the point at which pollen shed and silking reached 50% (Table 4). All four events were delayed in pollen shed by 10-40 GDU relative to control, in order Null<E1<E3<E2<E4, or across all 4 events together the pollen shed was delayed an average 25 GDU. All four events were delayed in silking by 2-38 GDU relative to control, in order Null<E1<E3<E2<E4, or across all four events together the silking delay averaged 21 GDU. The ASI was little changed, control (31 GDU) to the 4 events ranging from 23-34 GDU and averaging across all 4 events 27 GDU.

TABLE 4
Flowering Time Differences for ZM-BG1H1 OE Plants

| ZM-BG1 | 50% Silk | | | | 50% Shed | | | | ASI | |
|---|---|---|---|---|---|---|---|---|---|---|
| Geno | | Δ | | Δ | | | | Δ | Δ | Δ |
| type | Hr | (Hr) | GDU | (GDU) | Hr | (Hr) | GDU | (GDU) | (Hr) | (GDU) |
| Null | 1537 | 0 | 1418 | 0 | 1515 | 0 | 1387 | 0 | 22 | 31 |
| E1 | 1538 | 1 | 1420 | 2 | 1521 | 6 | 1397 | 10 | 17 | 23 |
| E3 | 1553 | 16 | 1436 | 18 | 1524 | 9 | 1402 | 15 | 29 | 34 |
| E2 | 1560 | 23 | 1445 | 27 | 1539 | 24 | 1422 | 35 | 21 | 23 |
| E4 | 1572 | 35 | 1456 | 38 | 1544 | 29 | 1427 | 40 | 28 | 29 |
| E-AII | 1555 | 18 | 1439 | 21 | 1532 | 17 | 1412 | 25 | 23 | 27 |

The number of hours (Hr) or of accumulated heat growing units (GDU) since planting, whereat 50% of the plants exhibited visible ear silk emergence or tassel floret extrusion. The value for 50% of the plants was estimated by interpolation from the line-graph plot of the cumulative silking or pollen shed plants in the observation plot. The differential in hours or GDU for each event relative to control null was calculated. The E-All value is for all four events together. The anthesis-to-silking (ASI) interval in both hours and GDU for null and each event shown on the right.

Plant and Ear Height: Plant and ear height were measured from the ground to the first tassel branch or ear node for each plant, on days 74 and 75 respectively, by which time all plants had flowered. Average first tassel branch heights for all 4 events were taller than control by 4.9-10.1 cm, in relative order E4>E2>E2>E3>Null, with all four events averaging 8.0 cm taller (3.2%, t-test p<1×10−4). Average ear node heights for 3 out of 4 events were taller than control, ranging from −1.3 to +7.5 cm, in relative order E4>E2>E1>Null>E3, and across all four events averaging 2.8 cm higher (2.1%, t-test p=0.0272). The ratio of first tassel branch height to ear node height was however similar, with control at 1.94, and the events ranging from 1.92-1.99 and averaging 1.96, indicating that the plant height relative to ear height was little changed. However, for this ratio the event order E3>E1>E2>Null>E4 did reverse relative to event order for plant or ear height, indicating a possible slight rise of ear node relative tassel heights among the tallest events.

### EXAMPLE 4

### Ear and Kernel Morphology

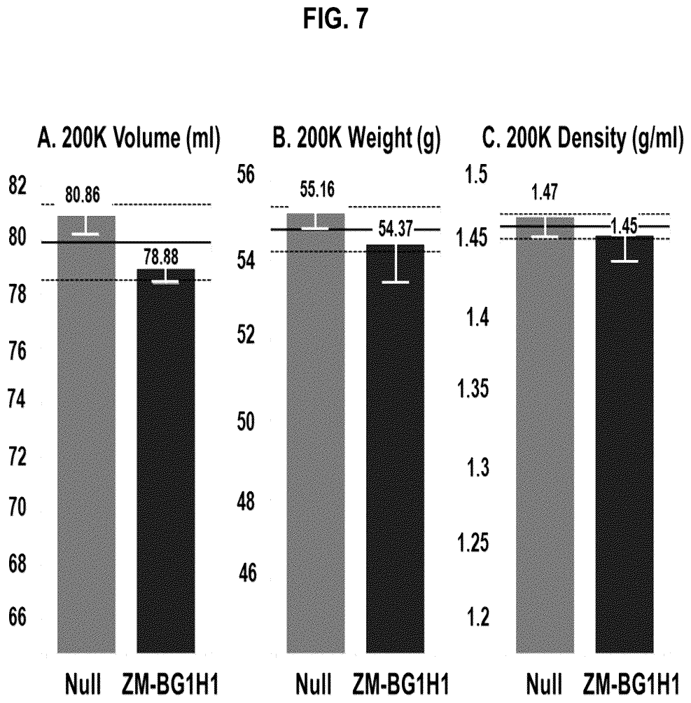

The same F1 hybrid seed sources used for planting the year 1 yield trials was evaluated for seed size and density by a combination of direct seed volume and weight measures. The seed volume, weight and density were compared between the control and the four transgenic ZM-BG1H1 OE transgene event lines (FIG. 7). Across all four events measured in three replicates the kernel volume averaged 2.5% lower than the control, and average kernel weight was 1.5% lower, and the kernel density was 1.4% lower (FIG. 7). The null hypothesis (no difference) is however not rejected at alpha 0.05 for each of these metrics. In contrast to the observation with the Os BG1 in rice, none of the four ZM-BG1H1 overexpressing events show increased seed size relative to control. This also indicates that the ZM-BG1H1 OE events hybrid yield trials did not benefit at planting from larger seeds than control seeds.

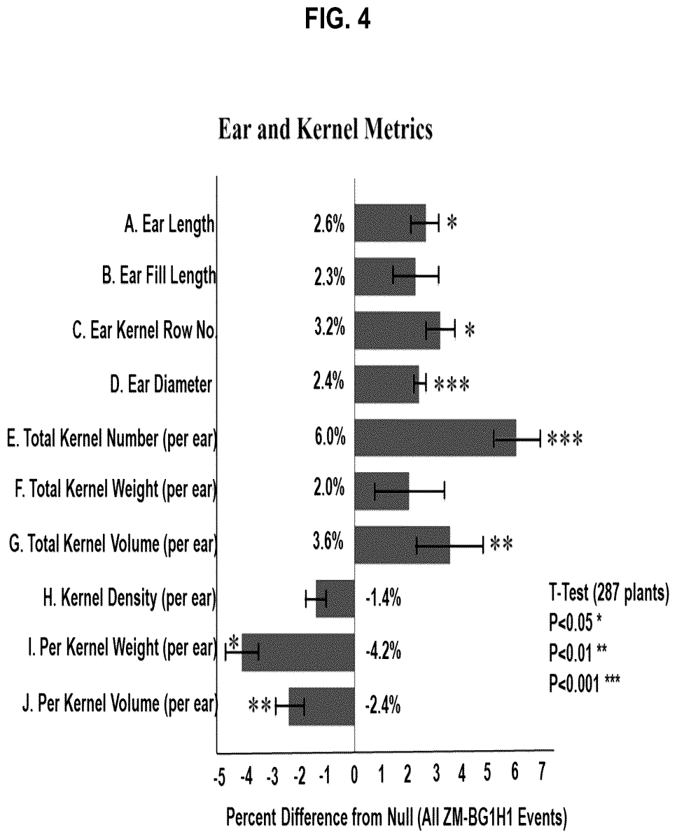

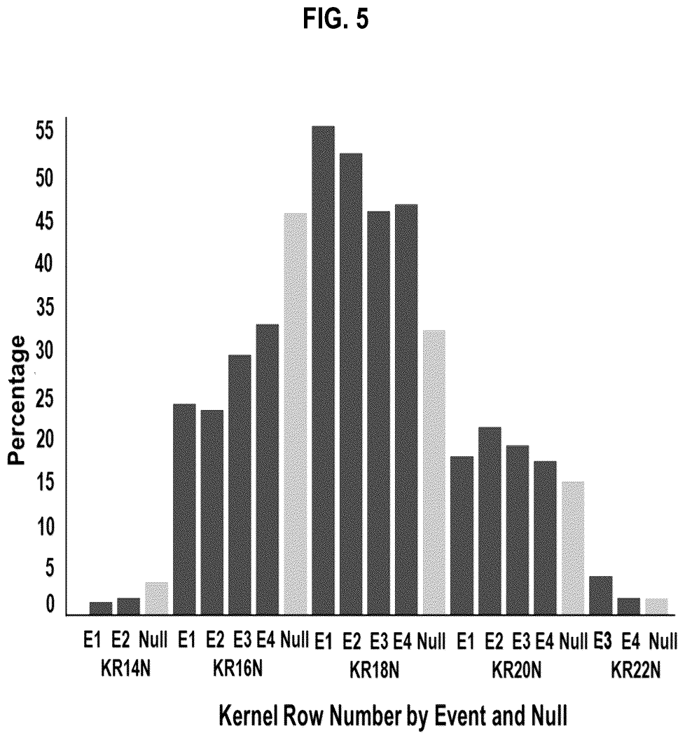

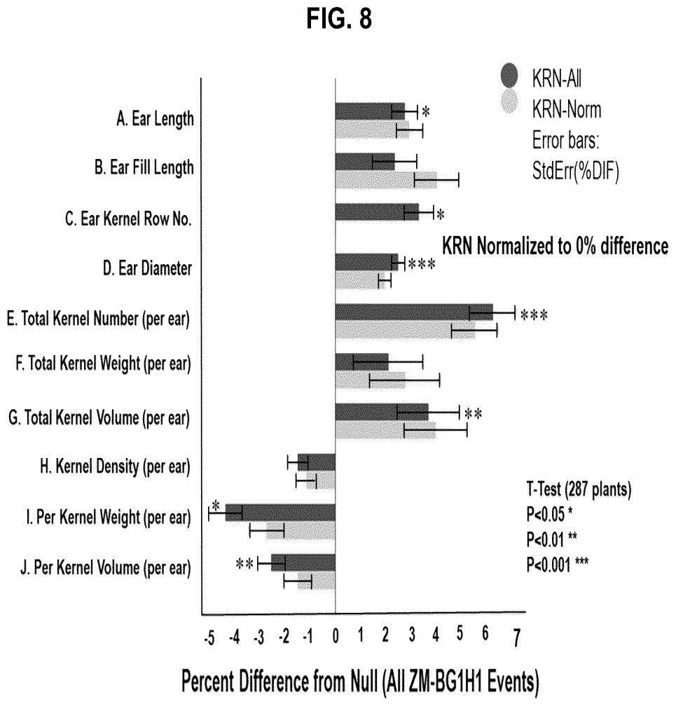

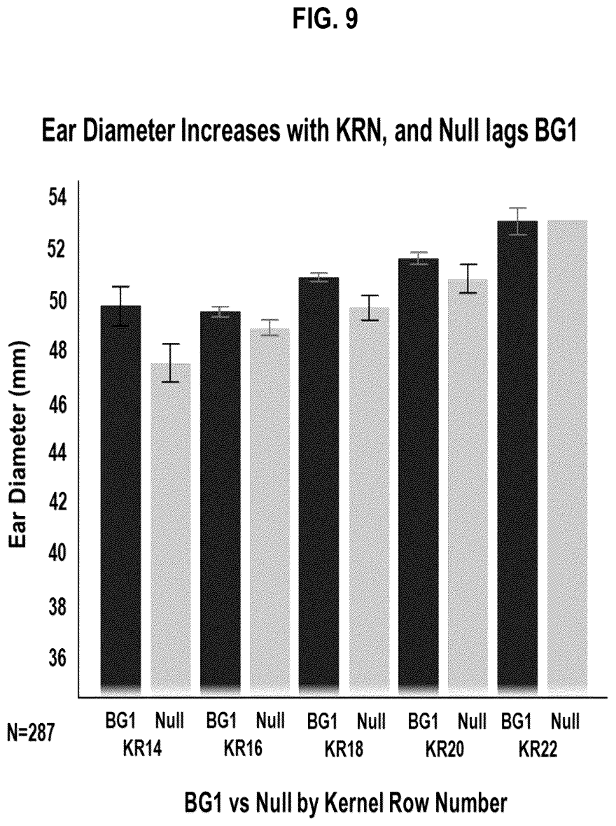

The observation plot ear and kernel data analysis are presented in FIG. 4. Across all four events per ear the total kernel number increased 6.0%, total kernel volume increased 3.6%, and total kernel weight increased 2.0%. Because each plant has only one ear, this increase in kernel weight reflects a yield gain per plant. Associated with this was a 2.6% increased ear length, a 2.3% increased ear filled length, and a 2.4% increased ear diameter. The average per kernel weight on each ear however decreased 4.2%, as did per kernel volume by 2.4%, leading to a slight 1.4% decline in per kernel density (FIG. 4). The ZM-BG1H1 OE plant ears for each of the four events showed increased average kernel row number (KRN), collectively across all events 17.86 KRN (ZM-BG1H1) versus 17.31 KRN (Control), a half row increase or 3.1%, with the t-test p-value 0.02. This upward KRN shift was observed for all four events, and the difference was most pronounced between 16 and 18 KRN (FIG. 5). Event E3 that had the largest KRN increase also had the largest field yield increase. Therefore, in considering the possibility that the 2.4% average increased ZM-BG1H1 OE yield may be primarily driven by this 3.1% increased of a half kernel row, and that the decrease in average ZM-BG1H1 OE kernel volume may relate to spatial constraints on it having a proportionally increased number of higher KRN ears, we compared the ear and kernel traits again but normalized for each discrete KRN value (FIG. 8). The results revealed that all the observed patterns of increase or decrease among ear or kernel traits observed in FIG. 4, persist in the roughly the same pattern and magnitude when comparing ears of the same KRN or all KRN together, with no statistically significant percentage differences (t-test P-values >0.1). Controlling for KRN did however nominally reduce the differences for ear diameter and total kernel number, as may be expected, because those two traits should increase with KRN. The ear diameter does increase with KRN for both ZM-BG1H1 OE and Control, yet the Null lagged behind ZM-BG1H1 OE at each KRN value in this sample (FIG. 9).

### EXAMPLE 5

### Promoter Analyses Among Native ZM-BG1H1 Homologs

OS-BG1 and BG1 homolog promoters were reported to possess auxin-response related motifs (Mishra et al 2017). A de novo search for conserved motifs found in proximal promoter, the first 1000 nts upstream from the ATG, for each of BG1 homologs across 5 species: ZM-BG1H1, OS-BG1, and BG1 homologs from Sorghum, Brachypodium and Setaria. Conserved motifs were searched in regions between ATG-TATA, and upstream from their shared TATA box to control for 5-UTR length variation affecting conserved motif relative offset positions. There exists a well-defined TATA box context CTATATCTTC in all the genes immediately upstream from the available 5′UTRs. There is also a conserved motif GCATTG in the 5′-UTRs amid additional 5′UTR sequence conservation. Five other motifs upstream from the TATA box were identified: CGCCAC, CCCGT, CACCC, GAAAT, and GGACG. Collectively all these seven elements are conserved in relative order, and they are within 360 nts from the TATA box of ZM-BG1H1-A1. There are other conserved motifs, but some have multiple copies and/or are in varied positions relative to these 7 conserved elements, reducing confidence in their relevance. Apart from the TATA box, the functions of the 6 other motifs are unknown. Nonetheless, 5 of these motifs overlap with enriched LDSS heptamers, and 2 have matches to regulatory elements in PLACE database. None of these however are known to be associated with auxin. Furthermore, the 5 auxin response motifs are not among or overlapping any of these 7 conserved motifs: ACTTTA, TGACG, CATATG were found in only some promoters; TGTGNN and NNGACA were found in multiple locations suggesting non-specificity; and CACGCAAT and KGTCCCAT were not found at all.

Subcellular Localization: The subcellular localization of ZM-BG1H1 protein was investigated to address the following two questions: (1) is the ZM-BG1H1 protein localized to the plasma membrane (PM) as was reported for OS-BG1; and (2) is the ZM-BG1H1 protein localized to the PM with ZM-GOS2 PRO ectopic expression. Maize protoplasts transfected with two color markers, RFP were used to illuminate the nucleus and normalize expression levels, and GFP, in fusion to ZM-BG1H1 protein or not, to probe ZM-BG1H1 cellular location. Control broad cellular localization of GFP, and the demarcation of the nucleus when RFP is nuclear targeted by an NLS (nuclear localization signal) was performed. microscopic images of protoplast transfected with various Promoter::GFP reporter gene fusions as labelled at bottom of figure. Most protoplast range 20-30 microns diameter. Green color emanates from the GFP reporter gene, and red color from the RFP reporter gene. GFP is preferentially located at the protoplast plasma membrane. GFP is fused to the N-terminus of ZM-BG1H1 and ectopically expressed with ZM-GOS2 PRO. The result demonstrated GFP is localized chiefly to the cell surface consistent with the PM. A related experiment was done except that the ZM-BG1H1 coding region is fused instead the N-terminus of GFP. The result is similar, indicating that the ZM-BG1H1 protein is itself capable of directing the GFP protein to the PM regardless of whether its N-terminus or C-terminus is occupied by the fused GFP protein. The native ZM-BG1H1 RO has very low expression, and in this protoplast experiment, it also is expressed at low levels, at least an order of magnitude lower, which required longer exposure to reveal the diffuse localization of untargeted GFP expression. The native ZM-BG1H1 promoter driving GFP::ZM-BG1H1 fusion expression produced too low an expression to clearly see any PM localization.

### EXAMPLE 6

### ZM-BG1H1 Allelic Variation

Structural allelic diversity of the ZM-BG1H1 locus was surveyed in a breeding germplasm using a combination of a small number of completed high quality public and proprietary genome drafts, and some lower quality genome and transcriptome assemblies for 582 inbred lines were investigated, distribution 47% SS and 53% NSS. The allelic sequence comparison was limited to the core gene region from 1000 bp Promoter/5UTR/ORF/3UTR, because larger regions around the gene may include more recombination events that may thus subdivide into more haplotypes, but less likely to represent functionally distinctive ZM-BG1H1 gene alleles. For homolog ZM-BG1H1, at least 5 major sequence variants with possibly 8-13 minor sequence variants total, were observed. The first five variants, which we here refer to as alleles, are represented by high-quality gene region sequences. The other more speculative sequence variants are based upon lower quality consensus sequences and are not completely sequenced in any one inbred line and are therefore not here elaborated. These five allelic sequences presented account for 93% of the germplasm lines surveyed. Alleles A1 and A2 are found almost exclusively in SS (stiff-stalk, usually female in hybrid production), and account collectively for about 44% of the germplasm surveyed. Alleles A3, A4 and A5 count for 49% of the genome surveyed and are almost entirely NSS (non-stiff-stalk, usually male in hybrid production) (Table 5). The other speculative lower-quality variants account for the remainder. There is no indication of any presence-absence variation (PAV) at this locus. A separate earlier analysis of 416 germplasm lines (63% shared with the 582-line survey set) also did not uncover any PAV.

TABLE 5
Maize Allelic Diversity and Heterotic
Group Relationships at ZM-BG1H1 Locus

| | % | % | Refer- | | |
|---|---|---|---|---|---|
| | AAID | AAID | ence | % | |
| | to | to | Inbred | All | Heterotic |
| Gene-Allele | OS_BG1 | SB_BG1 | Line | Lines | Grouping |
| ZM-BG1H1-A1 | 65.1 | 77.5 | B73 | 44.4 | SS |
| ZM-BG1H1-A2 | 66.9 | 80.3 | PH1V69 | | (97.3%) |
| ZM-BG1H1-A3 | 66.8 | 78.4 | PHTD5 | 26.4 | NSS |
| | | | | | (100%) |
| ZM-BG1H1-A4 | 66.4 | 78.6 | PH3KP | 16.8 | NSS |
| | | | | | (97.0%) |
| ZM-BG1H1-A5 | 66.4 | 80.1 | PHH9H | 5.5 | NSS |
| | | | | | (100%) |

Global amino acid identity (AAID) (determined by ClustalW algorithm alignments) of each of the five most common maize allelic ZM-BG1H1 variants to the rice BG1 or Sorghum bicolor BG1 homologs. A reference inbred name bearing each of the five maize alleles, and the percentages of all lines assessed with each allelic haplotype, and the percentage of those lines that are considered either stiff-stalk or non-stiff-stalk.

All five alleles presented include a complete open reading frame, with no premature truncations or obviously defective incomplete proteins. The nucleotide identities range from 94.8-99.3% in the CDS. The encoded proteins are all distinct, ranging from 95.4-99.4% AAID among themselves, and to OS-BG1 (65.1-66.9% AAID) and to sorghum SB-BG1 (XP 021314015.1) (77.5-80.3% AAID) (Table 5). There are 7 peptide regional differences among the alleles. In comparison to sorghum SB-BG1, at 3 of 7 locations, the loss of histidine in “MQSHQDL” in ZM-BG1H1A2(3), and losses of “APAP” and “YGHG” in ZM-BG1H1A1, these variations appear to be maize lineage-specific. CDS comparisons indicate additional synonymous codon variations, and that the ZM-BG1H1A1's “APAP” variation is likely an SSR. Each of the 7 variant peptide regions were compared to Poaceae BG1 homolog representatives. All 7 locations were also regionally variable among the cross-species Poaceae BG1 peptides, suggesting these variants are not likely disrupting critical conserved protein function. The patterns of variation among the seven regions across the five maize alleles suggests a history of multiple intra-genic/inter-allelic recombination events. The five ZM-BG1H1 alleles were also compared in the proximal 1000 nt promoter plus 5′UTR region. Both the 5′UTR and Promoter regions show many variations, including indels and point mutations. Yet, all five alleles possess the multi-species conserved TATA box (CTATATCTTC), and among the 6 other motifs found above to be shared across BG1 homologs from multiple species, all are also conserved across all these five alleles, suggesting these variations are not likely disrupting conserved promoter function as observed in the evaluations.

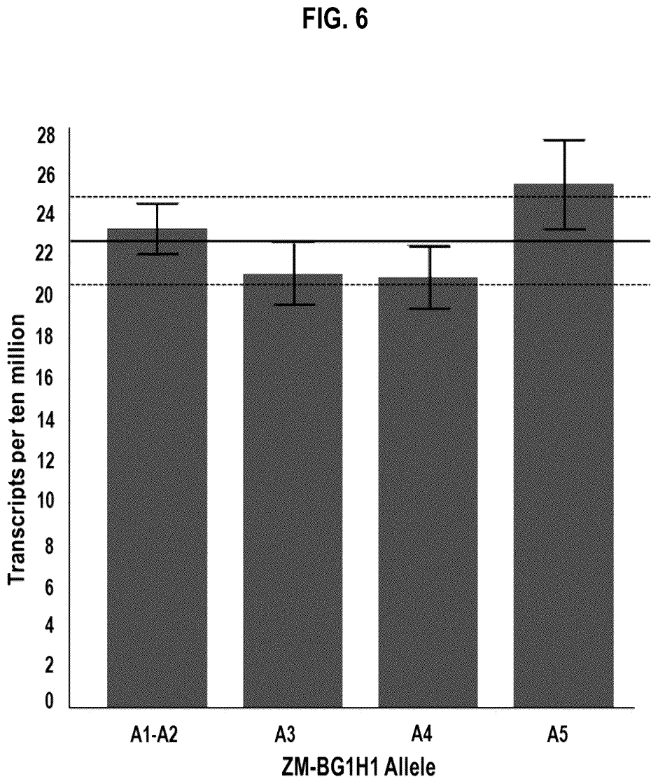

Allelic functional differences may manifest in gene expression differences. A set of 416 inbred lines was surveyed for V6 leaf tissue expression harvested late morning between 10-12 AM. Marker and pedigree analyses enabled inference of the likely IBD haplotypes. Often key lines in each IBD haplotype could be matched by allelic IIS sequences for the five alleles presented here, but one such inferred identity by descent haplotype often contained both A1 and A2 alleles, indicating flanking genetic markers alone may fail to accurately distinguish these two alleles. Leaf expression was detected for all alleles, but as noted above leaf-day expression is low, here ranging from 21.0 to 25.5 pptm, but with little observed variation between the haplotypes (FIG. 6). The inbred line PH184C containing the ZM-BG1H1-A3 allele, and the same line used for the transgene transformation in this experiment, was subjected to RNA profiling analyses using field grown samples. The plants were sampled across 11 tissues at stages V10, VT/R1, and R4, and under drought and well-watered conditions. The average expression for each tissue is shown in Figure S11. This experiment did not directly compare other lines or haplotypes, however it reveals that the ZM-BG1H1-A3 (NSS, PH184C) allele is expressed in all tissues, and in a tissue-spatial pattern consistent with the broader tissue survey done for ZM-BG1H1-A1 (SS, B73); for example immature ear expression is relatively high, but expression is low in leaves.

It was assessed whether the ZM-BG1H1 and ZM-BG1H2(3) loci associated with various genetic-phenotypic intervals (QTLs, GWAS, Breeding Values, etc.). Over 3000 maize public and internal genetic intervals were searched involving traits in categories classified as Yield, Kernel, Development, Architecture, Root, Fertility and Flowering. One set involved 1860 published and curated regions and another involved over 1180 internally computed QTL and GWAS associations. Remarkably few regions associated with either the ZM-BG1H1 or ZM-BG1H2(3) loci. Occasionally yield plant height and maturity regions overlapped ZM-BG1H1 and ZM-BG1H2(3), however overall and there was no concentration of regions for any trait at either locus, rather an apparent relative deficiency at these two loci. A statistical significance of this conclusion is difficult to determine given the heterogenous aggregated information involved.

Terms used in the claims and specification are defined as set forth below unless otherwise specified. It must be noted that, as used in the specification and the appended claims, the singular forms “a,” “an” and “the” include plural referents unless the context clearly dictates otherwise.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1-4. (canceled)

5. A method of increasing maize grain yield, the method comprising increasing the expression of a polynucleotide encoding an amino acid sequence that is at least 95% identical to an amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 in a maize plant, wherein the polynucleotide is operably linked to a heterologous regulatory element; and growing the maize plant in a crop growing environment.

6. The method of claim 5, wherein the regulatory element is a heterologous promoter.

7. The method of claim 5, wherein the regulatory element is a moderative constitutive heterologous promoter.

8. The method of claim 5, wherein the regulatory element is a maize GOS2 promoter.

9. The method of claim 5, wherein the grain yield is increased by at least 2.0 bu/acre compared to a control plant not comprising increased expression of the polynucleotide.

10-16. (canceled)

17. A method of increasing yield of a plant, the method comprising increasing the expression of a polynucleotide encoding an amino acid sequence that is at least 95% identical to an amino acid sequence selected from the group consisting of SEQ ID NOs: 1, 3, 5, 7, 9, 11, 13, 15, 17, 19, 21, 23, 25, 27, 29, 31, 33, 35, 41, 43, 45, 47, 49, 51, 53, and 55 in a plant, wherein the polynucleotide is operably linked to a heterologous regulatory element; and growing the plant in a crop growing environment.

18. The method of claim 17, wherein the plant is a monocot plant.

19. The method of claim 17, wherein the plant is selected from the group consisting of maize, rice, soybean, wheat, sorghum, barley, brassica, sugarcane, cotton, and alfalfa.

20. A maize plant comprising in its genome a stably integrated recombinant DNA expression cassette, wherein the expression cassette comprises a polynucleotide encoding an amino acid sequence that is at least 95% identical to an amino acid sequence selected from the group consisting of SEQ ID NOs: 1, 3, 5, 7, 9, 11, 13, 15, 17, 19, 21, 23, and 25 and wherein the maize plant exhibits increased yield, improved agronomic parameter or a combination thereof, compared to a control maize plant not comprising the recombinant DNA expression cassette.

21. The maize plant of claim 20, wherein the yield is increased by at least 2.0 bu/acre compared to the control maize plant.

22. The method of claim 5, wherein the field grain yield is increased by at least about 2.0, 2.5, 3.0, 3.5, 4.0, 4.5, or 5.0 bu/acre compared to the control maize plant.

23. The method of claim 5, wherein the maize plant is planted at a density of about 25,000; 30,000; 35,000; 40,000; 45,000; 50,000; 55,000 or 60,000 plants per acre.

24. The maize plant of claim 20, wherein the field grain yield is increased by at least about 2.0, 2.5, 3.0, 3.5, 4.0, 4.5, or 5.0 bu/acre compared to the control maize plant not comprising increased expression of the polynucleotide.

25. The maize plant of claim 20, wherein the maize plant is planted at a density of about 25,000; 30,000; 35,000; 40,000; 45,000; 50,000; 55,000 or 60,000 plants per acre.

26. The maize plant of claim 20, wherein the stably integrated recombinant DNA expression cassette further comprises a heterologous regulatory element operably linked to the polynucleotide.

27. The maize plant of claim 24, wherein the regulatory element is a heterologous promoter.

28. The maize plant of claim 24, wherein the regulatory element is a moderative constitutive heterologous promoter.

29. The maize plant of claim 20, wherein the improved agronomic parameter is drought tolerance or nutrient utilization

30. The method of claim 17, wherein the plant is maize.
